(19) United States
(12) Patent Application Publication
(10) Pub. No.: US 2024/0384282 A1
(43) Pub. Date: Nov. 21, 2024

(54) REGULATION OF ROOT DEVELOPMENT

(71) Applicant: UT-Battelle, LLC, Oak Ridge, TN (US)

(72) Inventors: Tao Yao, Oak Ridge, TN (US); Jin-Gui Chen, Oak Ridge, TN (US); Wellington Muchero, Oak Ridge, TN (US)

(21) Appl. No.: 18/667,301

(22) Filed: May 17, 2024

Related U.S. Application Data
(60) Provisional application No. 63/467,630, filed on May 19, 2023.

(51) Int. Cl. C12N 15/82

(52) U.S. Cl. CPC C12N 15/8227

(57) ABSTRACT

The present disclosure provides methods to genetically engineer plants by manipulating the expression of the PtrXB38 polypeptide to increase root development. Also provided are genetically engineer plants that can be obtained by the methods according to the present disclosure.

### CROSS REFERENCE TO RELATED APPLICATION

This application claims the benefit of priority from U.S. Provisional Application No. 63/467,630, filed on May 19, 2023, the entire content of which is incorporated herein by reference.

### STATEMENT REGARDING FEDERALLY SPONSORED RESEARCH OR DEVELOPMENT

This disclosure was made with government support under a research project supported by Contract No. DE-AC05-00OR22725 awarded by the U.S. Department of Energy. The government has certain rights in this invention.

### INCORPORATION BY REFERENCE OF SEQUENCE LISTING

The Sequence Listing in an XML format, named as 42947_4872_2_SequenceListing of 24 KB, created on May 15, 2024, and submitted to the United States Patent and Trademark Office via Patent Center, is incorporated herein by reference.

### BACKGROUND

Adventitious root (AR) formation and development have been recognized as one of the most important traits in woody species breeding programs (Miller, A. J. et al., Am J Bot, 98, 1389-414, 2011). Successful rooting is a prerequisite for the survival of clonally propagated woody species, such as Populus (Bannoud, F. et al., Frontiers in Plant Science, 12, 2021). In many clonally propagated species, AR formation starts from the dedifferentiation of a specified cell type, followed by the formation of root meristem and AR primordia resulting in the emergence of Ars (Bellini, C. et al., Annu Rev Plant Biol, 65, 639-66, 2014; Díaz-Sala, C., Plants, 9, 1789, 2020). These processes are tightly regulated by endogenous hormone signals and external nutrient status (Lakehal, A. et al., Physiologia Plantarum, 165, 90-100, 2019; De Almeida, M. R. et al., Trees, 31, 1377-1390, 2017). Although different approaches have been employed to identify genes involved in AR formation across multiple plant species, the number of functionally characterized regulators is limited (Li, S. W., Front Plant Sci, 12, 614072, 2021; Sun, P. et al., Int J Mol Sci, 20, 2019; Trupiano, D. et al., Planta, 238, 271-282, 2013). As such, identifying genetic engineering and breeding targets that can be used to improve these traits remains a critical pursuit.

### SUMMARY OF THE DISCLOSURE

Clonal propagation is a commonly utilized technique in horticulture, agriculture, and forestry because it is an economically efficient method. The formation and growth of adventitious roots are essential for the success of clonal propagation for perennial tree species (Díaz-Sala, C., Plants, 9, 1789, 2020). Populus is a model perennial woody species with extensive genomic and genetic resources (Tuskan et al., Science, 313, 1596-1604, 2006; Muchero et al., Proceedings of the National Academy of Sciences, USA, 115, 11573-11578, 2018). Taking advantage of Populus natural variation and transcriptomics data, the present disclosure identifies genetic determinants of plant growth and development and elucidate the underlying transcriptional regulatory networks using eQTL (expression quantitative trait loci) methods. The present disclosure identifies XB3 family protein 8 in Populus trichocarpa (PtrXB38), as a key hub gene of root development and plant hormone signaling in Populus. The present disclosure demonstrates that PtrXB38 regulates both stem-born and base-born adventitious root formation. Omics studies reveals that endogenous plant hormones especially auxin signaling play crucial roles in PtrXB38-mediated adventitious root formation. This disclosure provides a method to genetically engineer plants by manipulating the expression of PtrXB38 polypeptide to increase root development.

One aspect of the present disclosure is directed to a genetically modified plant, plant cell or plant tissue, comprising an exogenous nucleic acid which comprises a nucleotide sequence encoding a PtrXB38 polypeptide or a homolog thereof, wherein the genetically modified plant, plant cell or plant tissue displays an enhanced expression of the PtrXB38 polypeptide or the homolog thereof and an increase in root development compared to a wild-type plant, plant cell, or plant tissue.

In some embodiments, the nucleotide sequence has at least 90% sequence identity to the nucleotide sequence shown in SEQ ID NO: 1. In some embodiments, the PtrXB38 polypeptide or the homolog thereof has at least 90% sequence identity to the amino acid sequence shown in SEQ ID NO: 2.

In some embodiments, the exogenous nucleic acid is stably integrated into the plant genome. In some embodiments, the nucleotide sequence is operably linked to a heterologous promoter.

In some embodiments, the heterologous promoter is a constitutive promoter, an inducible promoter, or a native promoter. In some embodiments, the heterologous promoter is a 35S promoter.

In some embodiments, the plant is a monocot or a dicot.

In some embodiments, the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

In some embodiments, the plant is Populus. In some embodiments, the plant is Populus trichocarpa.

In some embodiments, the root development comprises adventitious root formation.

In some embodiments, the adventitious root formation requires auxin transport and signaling. In some embodiments, the adventitious root formation requires ethylene biosynthesis and signaling.

In some embodiments, the enhanced expression of the PtrXB38 polypeptide or the homolog thereof increases flavonoids in roots, thereby increasing the resilience of the plant roots to environmental stresses.

In some embodiments, the plant displays one or more of the following characteristics:
has improved plant defense against pathogens by increasing production of ethylene and jasmonates compared to a wild type plant;
has increased flooding tolerance compared to a wild type plant; and has increased carbon dioxide (CO₂) absorption or carbon dioxide (CO₂) sequestration compared to a wild type plant.

Another aspect of the disclosure is directed to a method of increasing root development in a plant, plant cell or plant tissue, comprising introducing into the plant, plant cell or plant tissue, an exogenous nucleic acid sequence encoding a PtrXB38 polypeptide or a homolog thereof, thereby obtaining a genetically modified plant, plant cell or plant tissue, wherein the genetically modified plant, plant cell or plant tissue displays an enhanced expression of the PtrXB38 polypeptide or the homolog thereof and an increase in root development compared to a wild-type plant, plant cell, or plant tissue.

Another aspect of this disclosure is directed to a method of improving plant defense against pathogens in a plant, plant cell or plant tissue comprising enhancing expression of a PtrXB38 polypeptide or a homolog thereof in the plant, plant cell or plant tissue, thereby increasing production of ethylene and jasmonates compared to a wild type plant.

Another aspect of this disclosure is directed to a method of increasing flooding tolerance in a plant, plant cell or plant tissue comprising enhancing expression of a PtrXB38 polypeptide or a homolog thereof in the plant, plant cell or plant tissue, thereby increasing root development compared to a wild type plant.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1T. Identification of PtrXB38 as a hotspot by eQTL mapping. (A) Leaf and xylem expression of PtrXB38 across P. trichocarpa population. (B-E) Manhattan plots and quantile-quantile plots showing the identification of PtrXB38 as a cis-eQTL hotspot in leaf (B, D) and xylem (C, E). Displayed are −log 10 (P values) for association by genomic position. Chromosome (Chr) 10 containing the associated single nucleotide polymorphisms (SNPs) in PtrXB38 locus is highlighted in green. Chromosomal location of PtrXB38 is marked with arrows. (F, G) Association of the expression of PtrXB38 with one of the SNPs at the PtrXB38 locus, Chr10:9762348, from leaf (F) or xylem (G) transcriptome data. Genotype 0_1 is heterozygous of reference and alternative, and genotype 1_1 is the homozygous alternative. (H-O) Manhattan plots and quantile-quantile plots showing association of the expression of SCZ, OBP4, TCP20 and SVP by genomic position. Chr10 containing the associated SNPs in PtrXB38 locus is highlighted in green. Chromosomal location of PtrXB38 is marked with arrows. (P-S) Association of the expression of SCZ (P), OBP4 (Q), TCP20 (R), and SVP (S) with one of the SNPs at the PtrXB38 locus, Chr10:9762348. Genotype 01 is heterozygous of reference and alternative, and genotype 11 is the homozygous alternative. (T) The TF-based PtrXB38 regulatory network from eQTL network. The expression profiles of the second layer of TFs were used as phenotypes in the eQTL analysis and the highly associated SNPs flanking genes were extracted as the first layer of regulatory genes. The brown nodes represent the first layer of TFs, and the blue nodes represent the second layer of TFs. The green and orange edges represent the eQTL regulatory relationship was obtained from leaf and xylem dataset, respectively.

FIGS. 2A-2J. Over-expression of PtrXB38 promotes adventitious root formation in poplar. (A) Morphological phenotypes of adventitious root formation in control and two PtrXB38-OE independent lines, OE-72 and OE-22. The arrows denote the adventitious roots developed from stem or leaf, bar=1 cm. (B) Schematic representation of aerial and below-ground root systems in control and PtrXB38-OE plants. (C, D) Aerial root numbers of control and two PtrXB38-OE lines on stem (C) and leaf (D). (E) Adventitious root numbers of control and two PtrXB38-OE lines. (F-J) Expression of adventitious root development related genes WOX5 (F), SHR (G), ARF15 (H), PIN1 (I), and AIL1 (J) in control and PtrXB38-OE micro-propagated cuttings. Bar charts represent mean±s.e.m (n≥3 independent plants), and different letters represent significant difference between groups (p≤0.05) determined by one-way ANOVA followed by Tukey's test.

FIGS. 3A-3D. Auxin signaling and transport is essential for PtrXB38-mediated adventitious root formation. (A) Log-fold changes of transcript levels of auxin biosynthesis, signaling, transport and response related genes in 12-day-old root samples of PtrXB38-OE versus control plants, based on RNA-seq results. (B) Log-fold changes of protein abundance of auxin response factor in 12-day-old root samples of PtrXB38-OE versus control plants, based on quantitative proteomics. (C) Root formation of PtrXB38-OE plants upon auxin transport inhibitor treatment. (D) Adventitious root number of PtrXB38-OE plants with 0.2 μM and 1 μM NPA treatments. Bar charts represent mean±s.e.m, and different letters represent significant difference between groups (P≤0.05) determined by one-way ANOVA followed by Tukey's test.

FIGS. 4A-4F. PtrXB38-regulated adventitious root development involves ethylene biosynthesis and signaling. (A) Log-fold changes of transcript levels of ethylene biosynthesis genes in 12-day-old root samples of PtrXB38-OE versus control plants, based on RNA-seq. (B) Log-fold changes of protein abundance of ethylene biosynthesis enzymes in 12-day-old root samples of PtrXB38-OE versus control plants, based on quantitative proteomics. (C) Root formation of PtrXB38-OE plants upon ethylene biosynthesis inhibitor AVG treatment at 5 μM. (D) Log-fold changes of transcription levels of ERF genes in 12-day-old root samples of PtrXB38-OE versus control plants, based on RNA-seq. (E) Root morphology of PtrXB38-OE plants upon AVG treatment at 1 μM. (F) Root biomass of PtrXB38-OE in (E). Bar charts represent mean±s.e.m, and different letters represent significant difference between groups (P≤0.05) determined by one-way ANOVA followed by Tukey's test.

FIGS. 5A-5J. PtrXB38 affects flavan-3-ols biosynthesis in poplar. (A) Changes in root color of PtrXB38-OE plants. (B) Anthocyanin accumulation in the roots of PtrXB38-OE plants. (C) Schematic diagram of phenylpropanoid pathway and flavonoid biosynthesis. (D-H) Metabolite abundance of shikimic acid (D), phenylalanine (E), catechin (F), epicatechin (G), and epigallocatechin (H) in PtrXB38-OE plants. (I) Log-fold changes of transcript levels of phenylpropanoid pathway and flavonoid biosynthetic genes in leaf samples of PtrXB38-OE plants. Data is retrieved from RNA-seq results. (J) Log-fold changes of protein abundance of phenylpropanoid pathway and flavonoid biosynthetic genes in leaf samples of PtrXB38-OE plants. Data is retrieved from quantitative proteomics results. Bar charts represent mean±s.e.m, and different letters represent significant difference between groups (P≤0.05) determined by one-way ANOVA followed by Tukey's test.

FIG. 6. Identification of PtrXB38 as a hotspot by eQTL mapping. Circos plot for eQTL hotspot of PtrXB38. The yellow lines show the connections between PtrXB38 and associated genes across different chromosomes. Colored boxes represent the different chromosomes.

FIGS. 7A-7B. Protein domains and tissue expression patterns of PtrXBAT genes. (A) Domain structures of XBAT family in Populus trichocarpa. Rice XB3 protein was used as a template. (B) Tissue expression patterns of XBAT gene family in Populus trichocarpa. The expression data of different tissues was obtained from Populus Gene Express Atlas.

FIG. 8. Expression levels of PtrXB38 in independent lines of PtrXB38-OE transgenic plants. Total RNA was extracted from leaves of one-month-old Populus plants for qRT-PCR analysis. Bar charts represent mean±s.e.m (n=3 independent plants).

FIGS. 9A-9B. Adventitious root numbers of PtrXB38-KO plants. (A) Mutation patterns of XB38 alleles in three independent CRISPR/Cas9 lines. The nucleotide length change of indel is indicated in red. The PtrXB38 gene is present as tandem duplicate in P. tremula x P. alba ‘INRA 717-1B4’ based on the draft genome assembly and hence four alleles are shown. ‘a’ represents the alternative subgenome copies, and ‘p’ represents the main/primary subgenome copies. (B) adventitious root number of control and PtrXB38-KO lines. Bar charts represent mean±s.e.m.

FIGS. 10A-10B. Overexpression of PtrXB38 alters the Populus transcriptome in roots. (A) Principal component analysis of the transcriptome data. X and Y axis show principal component 1 and principal component 2 that explain 26.8% and 25.6% of the total variance, respectively. N=12 data points. (B) Abundance profile of Potri.010G070800.6 transcript (annotated was PtrXB38). Bar charts represent mean±s.e.m, and different letters represent significant difference between groups (P≤0.05) determined by one-way ANOVA followed by Tukey's test.

FIG. 11. Expression of transcription factor family genes regulated by PtrXB38. Number of TF coding gene among DEGs from RNA-seq results were counted in 12-day-old root tissue.

FIGS. 12A-12B. Functional category enrichment of the DEGs. (A-B) GO annotation (A) and KEGG pathway (B) analyses for the DEGs in 12-day-old PtrXB38-OE root tissues.

FIGS. 13A-13B. Overexpression of PtrXB38 alters the Populus proteome in roots. (A) Principal component analysis of the proteome data. X and Y axis show principal component 1 and principal component 2 that explain 72% and 11.9% of the total variance, respectively. N=9 data points. (B) Abundance profile of Potri.010G070800.6 protein (annotated was PtrXB38). Bar charts represent mean±s.e.m, and different letters represent significant difference between groups (P≤0.05) determined by one-way ANOVA followed by Tukey's test.

FIG. 14. Functional category enrichment of the DEPs. Results from GO annotation analysis are shown.

FIGS. 15A-15B. Glycerolipid content in PtrXB38-OE transgenic plants. Metabolite abundance of glycerol (A), monogalactosylglycerol (B), monogalactosylglycerol-like (C), and monoglucosylglycerol (D). Bar charts represent mean±s.e.m, and different letters represent significant difference between groups (P≤0.05) determined by one-way ANOVA followed by Tukey's test.

FIGS. 16A-16B. Growth advantage of PtrXB38-OE plants. (A) PtrXB38-OE saplings were grown in tissue culture medium for two months. (B) Fresh weigh of control and PtrXB38-OE plants. Bar charts represent mean±s.e.m, and different letters represent significant difference between groups (P≤0.05) determined by one-way ANOVA followed by Tukey's test.

### DETAILED DESCRIPTION

### Definitions

Unless defined otherwise, all technical and scientific terms used herein have the same meaning as commonly understood by one of ordinary skill in the art to which this invention belongs.

As used herein, the term “about” refers to an approximately +/−10% variation from a given value.

The term “control plant” as used herein, refers to a plant of the same species that does not comprise the modification or modifications described in this disclosure. In some embodiments, the control plant is of the same variety. In some embodiments, the control plant is of the same genetic background. In some embodiments, the control plant is wild-type plant.

The term “adventitious root (AR) formation” as used herein, refers to a postembryonic organogenesis process induced by differentiated cells other than those specified to develop roots. Adventitious root formation is a key step in vegetative propagation by stem cuttings, and has been exploited in horticulture, agriculture, and forestry. Lateral and adventitious roots arise by cell divisions in localized areas in the pericycle in the root and vascular parenchyma cells in stem or leaf tissues. Adventitious roots, arising from the stem of the plants, are the main component of the mature root system of many plants. Their development can also be induced in response to adverse environmental conditions or stresses. Auxin is the major growth-promoting hormone for the initiation of lateral and adventitious root growth. It induces cells in the pericycle and parenchyma to dedifferentiate and enter initial cell divisions.

As used herein, the term “auxin response factors (ARFs)” refers to a family of transcription factors that play an important role of auxin regulation through their binding with auxin response elements.

As used herein, the term “ethylene response factors (ERFs)” refers to AP2/ERF superfamily proteins belonging to the largest family of transcription factors known to participate during multiple abiotic stress tolerance such as salt, drought, heat, and cold with well-conserved DNA-binding domain.

The term “exogenous” as used herein, refers to a substance or molecule originating or produced outside of an organism. The term “exogenous gene” or “exogenous nucleic acid molecule” as used herein, refers to a nucleic acid that codes for the expression of an RNA and/or protein that has been introduced (“transformed”) into a cell or a progenitor of the cell. An exogenous gene may be from a different species (and so a “heterologous” gene) or from the same species (and so a “homologous” gene), relative to the cell being transformed. A transformed cell may be referred to as a recombinant or genetically modified cell. An “endogenous” nucleic acid molecule, gene, or protein can represent the organism's own gene or protein as it is naturally produced by the organism.

The term “expression” refers to the process of converting genetic information of a polynucleotide into RNA through transcription, which is catalyzed by an enzyme, RNA polymerase and into protein, through translation of mRNA on ribosomes. Expression can be, for example, constitutive or regulated, such as, by an inducible promoter (e.g., lac operon, which can be triggered by Isopropyl β-D-1-thiogalactopyranoside (IPTG). Up-regulation or overexpression refers to regulation that increases the production of expression products (mRNA, polypeptide or both) relative to basal or native states, while inhibition or down-regulation refers to regulation that decreases production of expression products (mRNA, polypeptide or both) relative to basal or native states. Expression of a gene can be measured through a suitable assay, such as real-time quantitative reverse transcription polymerase chain reaction (qRT-PCR), Northern blot, transcriptome sequencing and Western blot.

The term “gene” as used herein, refers to a segment of nucleic acid that encodes an individual protein or RNA and can include both exons and introns together with associated regulatory regions such as promoters, operators, terminators, 5′ untranslated regions, 3′ untranslated regions, and the like.

The term “genetically modified” (or “genetically engineered” or “transgenic” or “cisgenic”) refers to a plant comprising a manipulated genome or nucleic acids. In some embodiments, the manipulation is the addition of exogenous nucleic acids to the plant. In some embodiments, the manipulation is changing the endogenous genes of the plant.

The term “homologous” refers to nucleic acids or polypeptides that are highly related at the level of nucleotide or amino acid sequence. Nucleic acids or polypeptides that are homologous to each other are termed “homologues”. The term “homolog” refers to a gene related to a second gene by descent from a common ancestral DNA sequence, therefore, the corresponding polynucleotide/polypeptide has a certain degree of homology, i.e., sequence identity (at least 10%, at least 20%, at least 40%, at least 60%, 65%, 66%, 68%, 70%, 75%, 80%, 86%, 88%, 90%, 92%, 95%, 97% or 99%). A “homolog” furthermore means that the function is equivalent to the function of the original gene.

Homologs of a given gene and corresponding or equivalent positions in the homologous genes or proteins can be determined by sequence alignment programs, e.g., including but not limited to, NCBI BLAST, ClustalW, DIAMOND, CS-BLAST, and MAFFT.

As used herein, the term “nucleic acid” has its general meaning in the art and refers to a coding or non-coding nucleic sequence. Nucleic acids include DNA (deoxyribonucleic acid) and RNA (ribonucleic acid) nucleic acids. Examples of nucleic acid thus include but are not limited to DNA, mRNA, tRNA, rRNA, tmRNA, miRNA, piRNA, snoRNA, and snRNA. Nucleic acids thus encompass coding and non-coding region of a genome (i.e., nuclear or mitochondrial or chloroplast).

The term “operably linked” refers to positioning of a regulatory region and a sequence to be transcribed in a nucleic acid so as to influence transcription or translation of such a sequence. For example, to bring a coding sequence under the control of a regulatory region, the translation initiation site of the translational reading frame of the polypeptide is typically positioned between one and about fifty nucleotides downstream of the promoter. A regulatory region can, however, be positioned as much as about 5,000 nucleotides upstream of the translation initiation site or about 2,000 nucleotides upstream of the transcription start site. A regulatory region typically comprises at least a core (basal) promoter.

The term “regulatory region” refers to a nucleic acid having nucleotide sequences that influence transcription or translation initiation and rate and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns and combinations thereof.

A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene (Fromm et al., The Plant Cell, 1:977-984 (1989)). The choice of regulatory regions to be included depends upon several factors, including, but not limited to, efficiency, selectability, inducibility, desired expression level and cell- or tissue-preferential expression. It is a routine matter for one of skill in the art to modulate the expression of a coding sequence by appropriately selecting and positioning regulatory regions relative to the coding sequence.

A “vector” is a replicon, such as a plasmid, phage or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses and retroviruses. Numerous vectors and expression systems are commercially available from such corporations as Novagen (Madison, Wis.), Clontech (Mountain View, Calif.), Stratagene (La Jolla, Calif.) and Invitrogen/Life Technologies (Carlsbad, Calif.).

PtrXB38 is also referred to as PtrXBAT35. PtrXB38 polypeptide is also referred to as PtrXBAT35 polypeptide. XB gene family is also referred to as XBAT gene family.

### Plants

There is no specific limitation on the plants that can be used in the methods of the present disclosure, as long as the plant is suitable to be transformed by a gene. The term “plant” as used herein, includes whole plants, plant tissues or plant cells. The plants that can be used for the methods and compositions of the present disclosure include various crops, flower plants or plants of forestry, etc. Specifically, the plants include, but are not limited to, dicotyledon, monocotyledon or gymnosperm.

In some embodiments, the methods and compositions of the present disclosure are also applicable over a broad range of plant species from the dicot genera Acer, Afzelia, Arabidopsis, Betula, Brassica, Eucalyptus, Fagus, Fraxinus, Glycine, Gossypium, Jatropha, Juglans, Linum, Lycopersicon, Medicago, Micropus, Populus, Prunus, Quercus, Salix, Solanum, Tectona and Trifolium; and the monocot genera Agrostis, Avena, Festuca, Hordeum, Lemna, Lolium, Milium, Miscanthus, Oryza, Panicum, Pennisetum, Phalaris, Phleum, Poa, Saccharum, Secale, Sorghum, Triticum, Zea and Zoysia; and the gymnosperm genera Abies, Picea and Pinus. In certain embodiments, the polynucleotides and vectors described herein can be used to transform a number of monocotyledonous and dicotyledonous plants and plant cell systems, wherein such plants are hybrids of different species.

### Expression Vectors

The polynucleotides and expression vectors described herein can be used to increase the expression of a PtrXB38 polypeptide or the homolog thereof in plants, plant cells or plant tissues.

The vectors provided herein can include origins of replication, scaffold attachment regions (SARs) and/or marker genes. A marker gene can confer a selectable phenotype on a plant cell. For example, a marker can confer biocide resistance, such as resistance to an antibiotic (e.g., kanamycin, G418, bleomycin or hygromycin) or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin or Flag-tag (Kodak, New Haven, Conn.) sequences can be expressed as a fusion with the encoded polypeptide.

Such tags can be inserted anywhere within the polypeptide, including at either the carboxyl or amino terminus. As described herein, plant cells can be transformed with a recombinant nucleic acid construct to express a polypeptide of interest.

### Promoters

A variety of promoters are available for use, depending on the degree of expression desired. For example, a broadly expressing promoter promotes transcription in many, but not necessarily all, plant tissues. Non-limiting examples of broadly expressing promoters that can be included in the nucleic acid constructs provided herein include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter and ubiquitin promoters such as the maize ubiquitin-1 promoter.

In some embodiments, the promoter to drive expression of genes of interest is a constitutive promoter. In some embodiments the constitutive promoter is selected from the group consisting of a ubiquitin promoter, a cauliflower mosaic virus (CaMV) 35S promoter, an actin promoter, a peanut chlorotic streak caulimovirus promoter, a Chlorella virus methyltransferase gene promoter, a full-length transcript promoter form figwort mosaic virus, a pEMU promoter, a MAS promoter, a maize H3 histone promoter and an Agrobacterium gene promoter.

In some embodiments, the promoter to drive expression of genes of interest is a regulated promoter. In some embodiments the regulated promoter is selected from the group consisting of a stress induced promoter, chemical-induced promoter, a light induced promoter, a dark-induced promoter, and a circadian-clock controlled promoter.

Some suitable regulatory regions initiate transcription, only or predominantly, in certain cell types. For instance, promoters active in photosynthetic tissue confer transcription in green tissues such as leaves and stems. Examples of such promoters include the ribulose-1,5-bisphosphate carboxylase (RbcS) promoters such as the RbcS promoter from eastern larch (Larix laricina), the pine chlorophyll a/b binding-6 (cab6) promoter (Yamamoto et al., 1994, Plant Cell Physiol., 35:773-778), the chlorophyll a/b binding-1 (Cab-1) promoter from wheat (Fejes et al., 1990, Plant Mol. Biol., 15:921-932), the chlorophyll a/b binding-1 (CAB-1) promoter from spinach (Lubberstedt et al., 1994, Plant Physiol., 104:997-1006), the cab IR promoter from rice (Luan et al., 1992, Plant Cell, 4:971-981), the pyruvate orthophosphate dikinase (PPDK) promoter from corn (Matsuoka et al., 1993. Proc. Natl. Acad. Sci. USA, 90:9586-9590), the tobacco light-harvesting complex of photosystem (Lhcb1*2) promoter (Cerdan et al., 1997, Plant Mol. Biol., 33:245-), the Arabidopsis SUC2 sucrose-H+ symporter promoter (Truernit et al., 1995, Planta, 196:564-570) and thylakoid membrane protein promoters from spinach (psaD, psaF, psaE, PC, FNR, atpC, atpD, cab, rbcS).

In some embodiments, promoters of the instant application comprise inducible promoters. Inducible promoters confer transcription in response to external stimuli such as chemical agents or environmental stimuli. For example, inducible promoters can confer transcription in response to hormones such as gibberellic acid or ethylene or in response to light, nitrogen, shade or drought.

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

A 5′ untranslated region (UTR) can be included in nucleic acid constructs described herein. A 5′ UTR is transcribed but is not translated and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a vector, e.g., introns, enhancers, upstream activation regions, transcription terminators and inducible elements. Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

Techniques for introducing nucleic acids into monocotyledonous and dicotyledonous plants, plant cells or plant tissues are known in the art and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863, incorporated herein by reference in their entirety. If a cell or tissue culture is used as the recipient tissue for transformation, plants can be regenerated from transformed cultures if desired, by techniques known to those skilled in the art. See, e.g., Niu et al., 2000. Plant Cell Rep. V19:304-310; Chang and Yang, 1996, Bot. Bull. Acad. Sin., V37:35-40; and Han et al., 1999, Biotechnology in Agriculture and Forestry, V44:291 (ed. by Y. P. S. Bajaj), Springer-Vernag.

Disclosed herein are plants, plant cells and plant tissues genetically modified by introduction of an exogenous nucleic acid, e.g., provided in an expression vector, thereby resulting in genetically modified plants, plant cells and plant tissues that display increased root development.

Typically, genetically modified plant cells used in methods described herein constitute part or all of a whole plant. Such plants can be grown in a manner suitable for the species under consideration, either in a growth chamber, a greenhouse or in a field. Genetically modified plants can be bred as desired for a particular purpose, e.g., to introduce a recombinant nucleic acid into other lines, to transfer a recombinant nucleic acid to other species or for further selection of other desirable traits. Progeny includes descendants of a particular plant or plant line provided the progeny inherits the transgene. Progeny of a plant include seeds formed on F1, F2, F3, F4, F5, F6 and subsequent generation plants or seeds formed on BC1, BC2, BC3 and subsequent generation plants or seeds formed on F1BC1, F1BC2, F1BC3 and subsequent generation plants. Seeds produced by a genetically modified plant can be grown and then selfed (or outcrossed and selfed) to obtain seeds homozygous for the nucleic acid construct. Alternatively, genetically modified plants can be propagated vegetatively for those species amenable to such techniques.

In some embodiments, the genetically modified plant, plant cell, or plant tissue, comprises an exogenous nucleic acid which comprises a nucleotide sequence encoding a PtrXB38 polypeptide or a homolog thereof, wherein the genetically modified plant, plant cell or plant tissue displays an enhanced expression of the PtrXB38 polypeptide or the homolog thereof and an increase in root development compared to a wild-type plant, plant cell, or plant tissue.

By “increased expression” it means the expression of the PtrXB38 polypeptide or the homolog thereof is increased as compared to a control plant, plant cell or plant tissue (e.g., a wild type plant, plant cell or plant tissue without the genetic modification, or a plant cell or plant tissue introduced with a control vector without the nucleotide sequence encoding the PtrXB38 polypeptide or the homolog thereof). The expression of the PtrXB38 polypeptide or the homolog thereof can be measured based on the gene expression (e.g., mRNA level) or the protein production (protein level). The extent of increase can be at least 50%, 100% (2 fold), 3 fold, 4 fold, 5 fold, 10 fold, 15 fold, or greater. In some embodiments, the increase is at least 2 fold. In some embodiments, the increase is at least 5 fold. In some embodiments, the increase is at least 10 fold.

In some embodiments, the root development comprises adventitious root formation. By “increase in root development”, it can include increase in root numbers and/or increase in root length from a genetically modified plant, plant cell or plant tissue, as compared to a control plant, plant cell or plant tissue without the genetic modification. The extent of increase, e.g., in the number of roots, can be at least 20%, 30%, 40%, 50%, 100% (2 fold), 3 fold, 4 fold, 5 fold, 10 fold, 15 fold, or greater. In some embodiments, the increase is at least 2 fold. In some embodiments, the increase is at least 5 fold. In some embodiments, the increase is at least 10 fold.

In some embodiments, the nucleotide sequence encoding a PtrXB38 polypeptide or a homolog thereof is set forth in SEQ ID NO: 1. In some embodiments, the nucleotide sequence has at least 90% sequence identity to the nucleotide sequence shown in SEQ ID NO: 1. In some embodiments, the nucleotide sequence has at least 91% sequence identity to the nucleotide sequence shown in SEQ ID NO: 1. In some embodiments, the nucleotide sequence has at least 92% sequence identity to the nucleotide sequence shown in SEQ ID NO: 1. In some embodiments, the nucleotide sequence has at least 93% sequence identity to the nucleotide sequence shown in SEQ ID NO: 1. In some embodiments, the nucleotide sequence has at least 94% sequence identity to the nucleotide sequence shown in SEQ ID NO: 1. In some embodiments, the nucleotide sequence has at least 95% sequence identity to the nucleotide sequence shown in SEQ ID NO: 1. In some embodiments, the nucleotide sequence has at least 96% sequence identity to the nucleotide sequence shown in SEQ ID NO: 1. In some embodiments, the nucleotide sequence has at least 97% sequence identity to the nucleotide sequence shown in SEQ ID NO: 1. In some embodiments, the nucleotide sequence has at least 98% sequence identity to the nucleotide sequence shown in SEQ ID NO: 1. In some embodiments, the nucleotide sequence has at least 99% sequence identity to the nucleotide sequence shown in SEQ ID NO: 1.

In some embodiments, the nucleotide sequence encoding a PtrXB38 polypeptide or a homolog thereof is a genomic DNA sequence set forth as in SEQ ID NO: 3.

In some embodiments, the PtrXB38 polypeptide or the homolog thereof has an amino acid sequence as laid out in SEQ ID NO: 2. In some embodiments, the PtrXB38 polypeptide or the homolog thereof has at least 90% sequence identity to the amino acid sequence shown in SEQ ID NO: 2. In some embodiments, the PtrXB38 polypeptide or the homolog thereof has at least 91% sequence identity to the amino acid sequence shown in SEQ ID NO: 2. In some embodiments, the PtrXB38 polypeptide or the homolog thereof has at least 92% sequence identity to the amino acid sequence shown in SEQ ID NO: 2. In some embodiments, the PtrXB38 polypeptide or the homolog thereof has at least 93% sequence identity to the amino acid sequence shown in SEQ ID NO: 2. In some embodiments, the PtrXB38 polypeptide or the homolog thereof has at least 94% sequence identity to the amino acid sequence shown in SEQ ID NO: 2. In some embodiments, the PtrXB38 polypeptide or the homolog thereof has at least 95% sequence identity to the amino acid sequence shown in SEQ ID NO: 2. In some embodiments, the PtrXB38 polypeptide or the homolog thereof has at least 96% sequence identity to the amino acid sequence shown in SEQ ID NO: 2. In some embodiments, the PtrXB38 polypeptide or the homolog thereof has at least 97% sequence identity to the amino acid sequence shown in SEQ ID NO: 2. In some embodiments, the PtrXB38 polypeptide or the homolog thereof has at least 98% sequence identity to the amino acid sequence shown in SEQ ID NO: 2. In some embodiments, the PtrXB38 polypeptide or the homolog thereof has at least 99% sequence identity to the amino acid sequence shown in SEQ ID NO: 2.

PtrXB38 belongs to a nine-member protein family. In some embodiments, the homologs of PtrXB38 include the other eight members: PtrXB31, PtrXB32, PtrXB33, PtrXB34, PtrXB35, PtrXB36, PtrXB37 and PtrXB39. These homologs share same protein domains (ANK domain and RING domain). In some embodiments, the PtrXB31 polypeptide has an amino acid sequence as laid out in SEQ ID NO: 4 which has 13.73% sequence identity to the amino acid sequence shown in SEQ ID NO: 2. In some embodiments, the PtrXB32 polypeptide has an amino acid sequence as laid out in SEQ ID NO: 5 which has 13.40% sequence identity to the amino acid sequence shown in SEQ ID NO: 2. In some embodiments, the PtrXB32 polypeptide is excluded from the homologs of PtrXB38 for purposes of this disclosure. In some embodiments, the PtrXB33 polypeptide has an amino acid sequence as laid out in SEQ ID NO: 6 which has 14.78% sequence identity to the amino acid sequence shown in SEQ ID NO: 2. In some embodiments, the PtrXB34 polypeptide has an amino acid sequence as laid out in SEQ ID NO: 7, which has 15.38% sequence identity to the amino acid sequence shown in SEQ ID NO: 2. In some embodiments, the PtrXB35 polypeptide has an amino acid sequence as laid out in SEQ ID NO: 8 which has 15.47% sequence identity to the amino acid sequence shown in SEQ ID NO: 2. In some embodiments, the PtrXB36 polypeptide has an amino acid sequence as laid out in SEQ ID NO: 9 which has 13.61% sequence identity to the amino acid sequence shown in SEQ ID NO: 2. In some embodiments, the PtrXB37 polypeptide has an amino acid sequence as laid out in SEQ ID NO: 10 which has 51.90% sequence identity to the amino acid sequence shown in SEQ ID NO: 2. In some embodiments, the PtrXB39 polypeptide has an amino acid sequence as laid out in SEQ ID NO: 11 which has 73.28% sequence identity to the amino acid sequence shown in SEQ ID NO: 2.

In some embodiments, the exogenous nucleic acid is stably integrated into the plant genome. Stably transformed cells typically retain the introduced nucleic acid with each cell division. The stably transformed genetically modified plants, plant cells or plant tissue can be useful in the methods described herein.

In some embodiments, the nucleotide sequence is operably linked to a heterologous promoter. The heterologous promoter can be a constitutive promoter, an inducible promoter, or a native promoter. In some embodiments, the heterologous promoter is a 35S promoter.

In some embodiments, the plant is a monocot or a dicot. In some embodiments, genetically modified plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia. In some embodiments, the plant is Populus. In some embodiments, the plant is Populus trichocarpa.

### Phenotypes of the Genetically Modified Plants

In some embodiments, the genetically modified plant, plant cell or plant tissue displays one or more of the following characteristics: has increased root development compared to a wild type plant, plant cell or plant tissue; has improved plant defense against pathogens by increasing production of ethylene and jasmonates compared to a wild type plant, plant cell or plant tissue; has increased flooding tolerance compared to a wild type plant; and has increased carbon dioxide (CO₂) absorption or carbon dioxide (CO₂) sequestration compared to a wild type plant, plant cell or plant tissue.

In some embodiments, the root development comprises adventitious root formation. By “increase in root development”, it can include increase in root numbers and/or increase in root length from a genetically modified plant, plant cell or plant tissue, as compared to a control plant, plant cell or plant tissue without the genetic modification. The extent of increase, e.g., in the number of roots, can be at least 20%, 30%, 40%, 50%, 100% (2 fold), 3 fold, 4 fold, 5 fold, 10 fold, 15 fold, or greater. In some embodiments, the increase is at least 2 fold. In some embodiments, the increase is at least 5 fold. In some embodiments, the increase is at least 10 fold.

In some embodiments, the genetically modified plant, plant cell or plant tissue disclosed herein displays improved plant defense against pathogens by increasing production of ethylene and jasmonates compared to a wild type plant, plant cell or plant tissue. Pathogens can include, for example, Pseudomonas syringae pv tomato DC3000. Production of ethylene and jasmonates can be determined by using known methodology, for example, laser-based photoacoustic detector and liquid chromatography tandem mass spectrometry (LC-MS/MS), respectively. The extent of increase can be, e.g., at least 10%, at least 20%, at least 30%, at least 40%, at least 50%, at least 75%, at least 100%, or greater.

In some embodiments, the genetically modified plant, plant cell or plant tissue disclosed herein displays improved flooding tolerance compared to a wild type plant, plant cell or plant tissue. Flooding tolerance can be achieved by gas exchange under flooding conditions, and thus increased flooding tolerance can be determined based on increased gas exchange under flooding conditions, as illustrated in the Examples hereinbelow. The extent of increase can be, e.g., at least 10%, at least 20%, at least 30%, at least 40%, at least 50%, at least 75%, at least 100%, or greater.

In some embodiments, the genetically modified plant, plant cell or plant tissue disclosed herein displays increased flavonoids in roots, thereby increasing the resilience of the plant roots to environmental stresses compared to a wild type plant, plant cell or plant tissue. The levels of flavonoids in roots can be measured by known methodologies, e.g., Gas Chromatography-Mass Spectrometry (GC-MS). The extent of increase can be, e.g., at least 10%, at least 20%, at least 30%, at least 40%, at least 50%, at least 75%, at least 100%, or greater.

Flavan-3-ols, such as catechin and epicatechin, are crucial compounds for plants to adapt to biotic and abiotic stresses. In some embodiments, three flavan-3-ols are significantly increased in the roots of genetically modified plants. In some embodiments, PtrXB38 affects flavonoid biosynthesis in roots. In some embodiments, the higher abundances of flavonoids increase the resilience of the genetically modified plant roots to environmental stresses.

### Methods

In some embodiments, disclosed herein is a method of increasing root development in a plant, plant cell or plant tissue. The method comprises introducing into the plant, plant cell or plant tissue, an exogenous nucleic acid sequence encoding a PtrXB38 polypeptide or a homolog thereof, thereby obtaining a genetically modified plant, plant cell or plant tissue, wherein the genetically modified plant, plant cell or plant tissue displays an enhanced expression of the PtrXB38 polypeptide or the homolog thereof and an increase in root development compared to a wild type plant, plant cell, or plant tissue.

In some embodiments, disclosed herein is a method of improving plant defense against pathogens in a plant, plant cell or plant tissue comprising enhancing expression of a PtrXB38 polypeptide or a homolog thereof in the plant, plant cell or plant tissue, thereby increasing production of ethylene and jasmonates compared to a wild type plant.

In some embodiments, disclosed herein is a method of increasing flooding tolerance in a plant, plant cell or plant tissue comprising enhancing expression of a PtrXB38 polypeptide or a homolog thereof in the plant, plant cell or plant tissue, thereby increasing root development compared to a wild type plant.

In some embodiments, disclosed herein is a method of increasing carbon dioxide (CO₂) absorption or carbon dioxide (CO₂) sequestration in a plant, plant cell or plant tissue comprising enhancing expression of a PtrXB38 polypeptide or a homolog thereof in the plant, plant cell or plant tissue, thereby increasing root development compared to a wild type plant.

In any of the methods, enhanced expression of a PtrXB38 polypeptide or a homolog thereof in a plant, plant cell or plant tissue can be achieved by introducing an exogenous nucleic acid encoding the PtrXB38 polypeptide or a homolog thereof, as described herein, into the plant, plant cell or plant tissue.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: eQTL Mapping

Whole-genome resequencing and variant calling for the 917 P. trichocarpa individuals was performed as described previously (Yates, T. B. et al., Genome Biol Evol 13, 1-14, 2021). The single nucleotide polymorphism (SNP) and indel dataset is available at Bioenergy Center website. A total of 390 and 444 RNA-Seq samples in leaf and xylem, respectively, were used to perform eQTL analysis. Prepublication RNA-seq data was made available for this study courtesy of the Center for Bioenergy Innovation and the Joint Genome Institute. Briefly, leaf and xylem tissue were collected in 2015 from 4-year-old trees at the Clatskanie, OR field site and processed as described previously (Zhang, J. et al., New Phytologist 220, 502-516, 2018). Reads were aligned to the Populus trichocarpa v.3.1 reference (Tuskan, G. A. et al., Science 313, 1596-604, 2006) using STAR (Dobin, A. et al., Bioinformatics 29, 15-21, 2013). Fragments per kilobase of transcript per million mapped reads (FPKM) counts were then obtained for each genotype, resulting in a genotype-transcript matrix. The gene expression profiles were then used as phenotypes in GWAS analyses using EMMAX (Zhou, X. & Stephens, M., Nat Genet 44, 821-4, 2012). SNPs and indels with minor allele frequencies≥0.05 identified from whole-genome resequencing were used for this analysis. To account for multiple testing, the Bonferroni corrected threshold, P≤0.05/8253066=6.06×10⁻⁹, was used to determine statistical significance.

### Example 2: Genetic Mapping of the PtrXB38 Gene

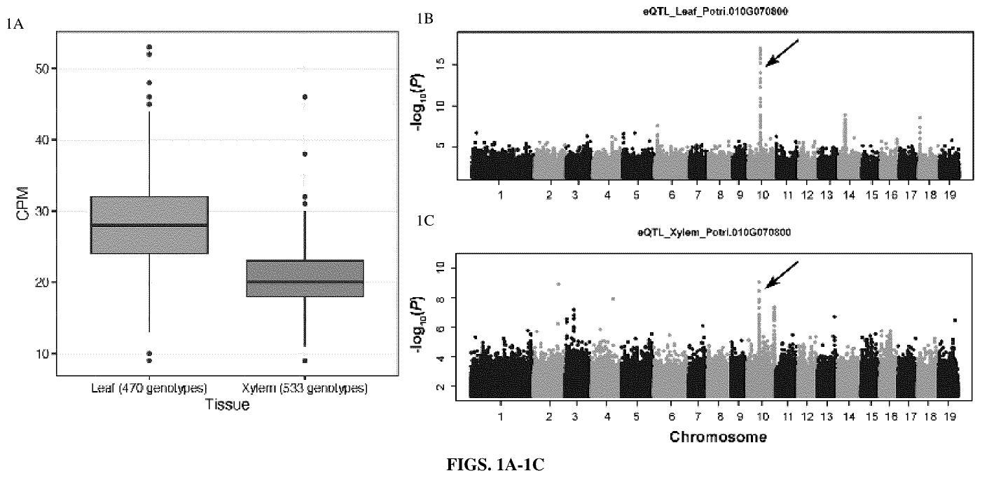

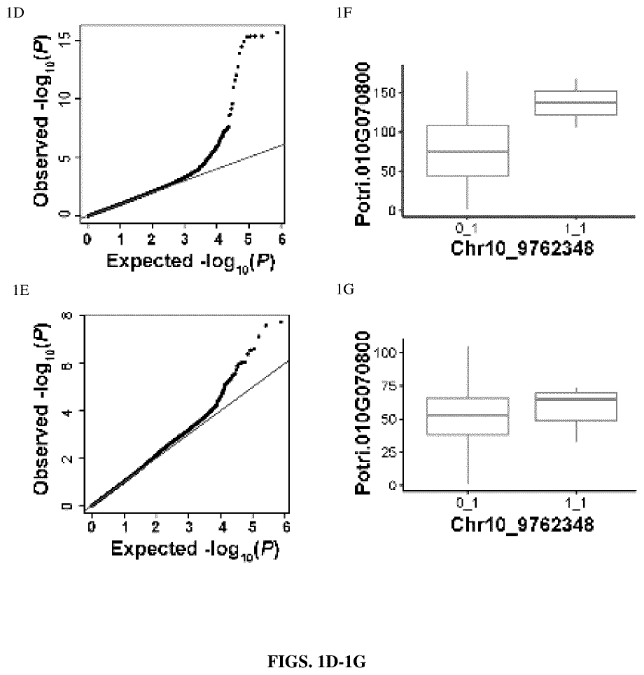

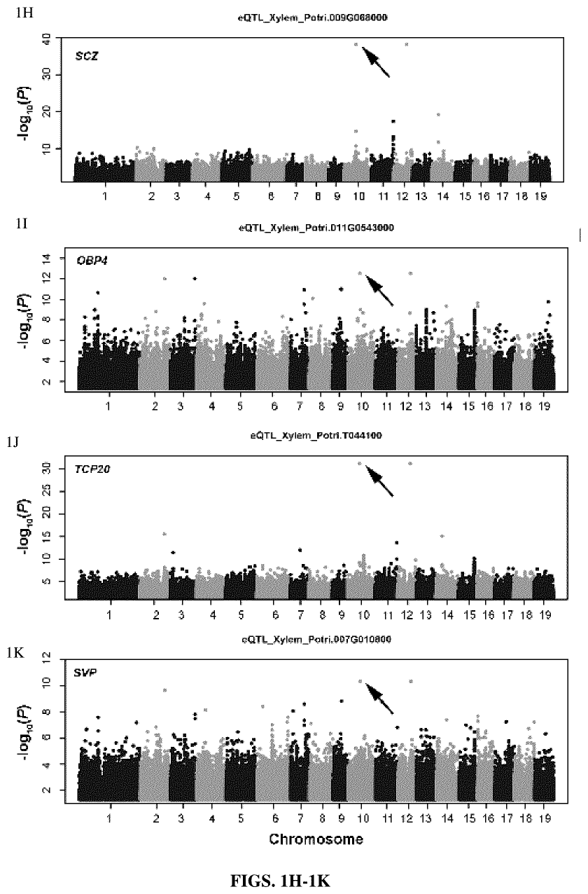

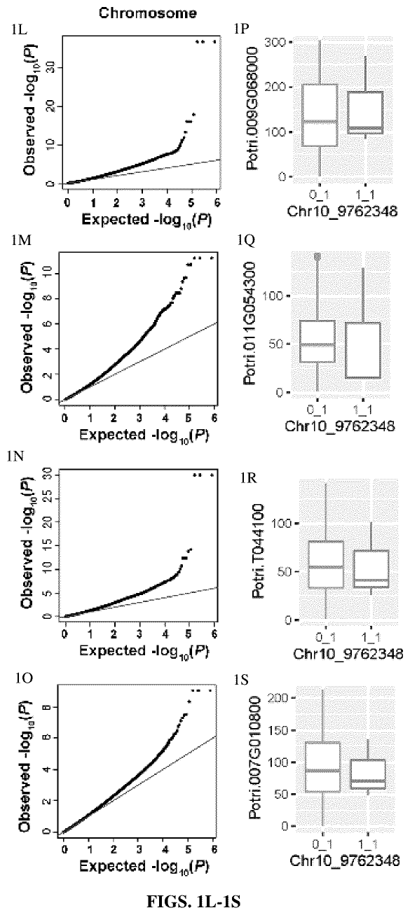

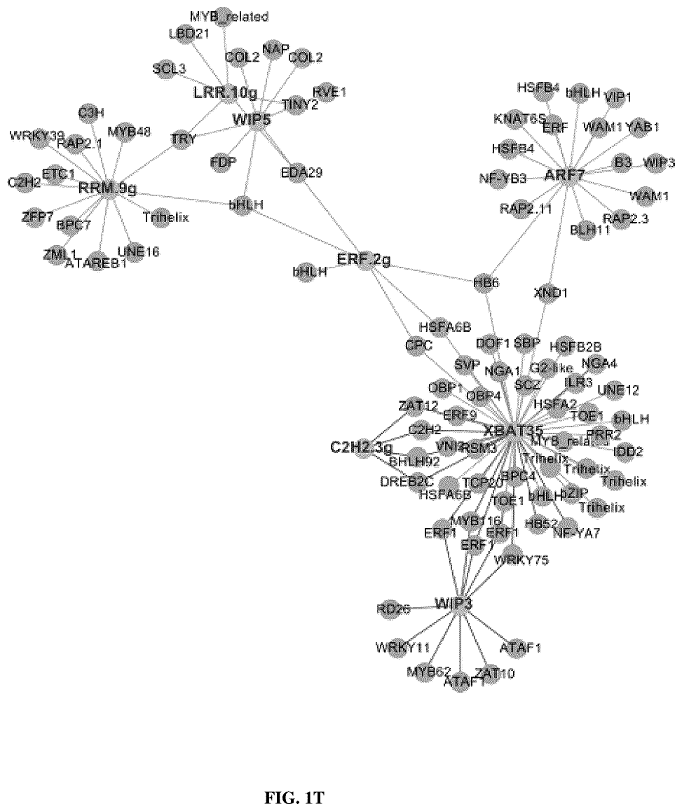

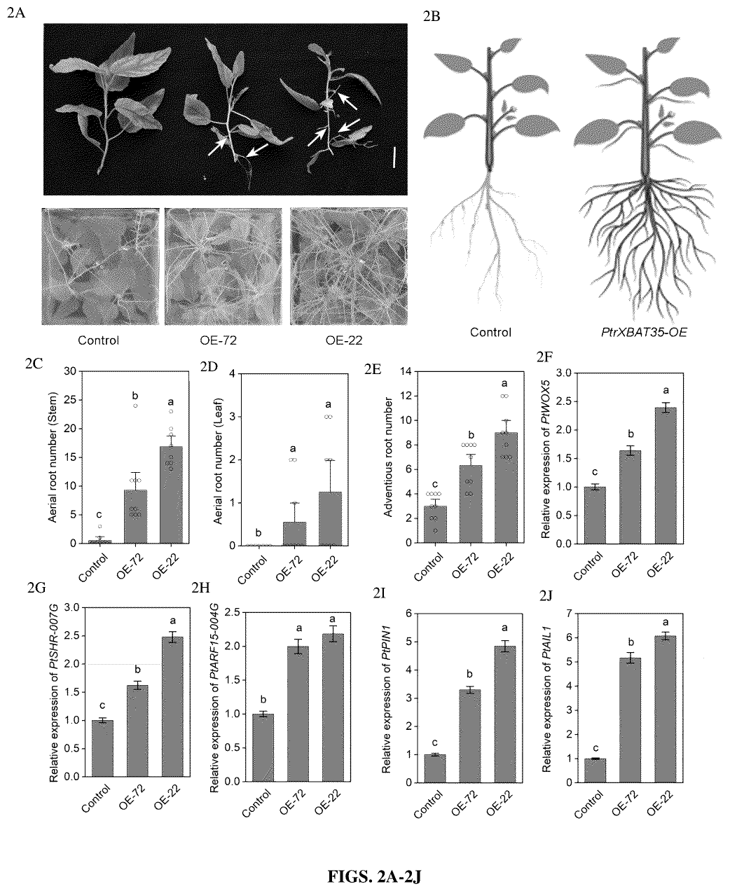

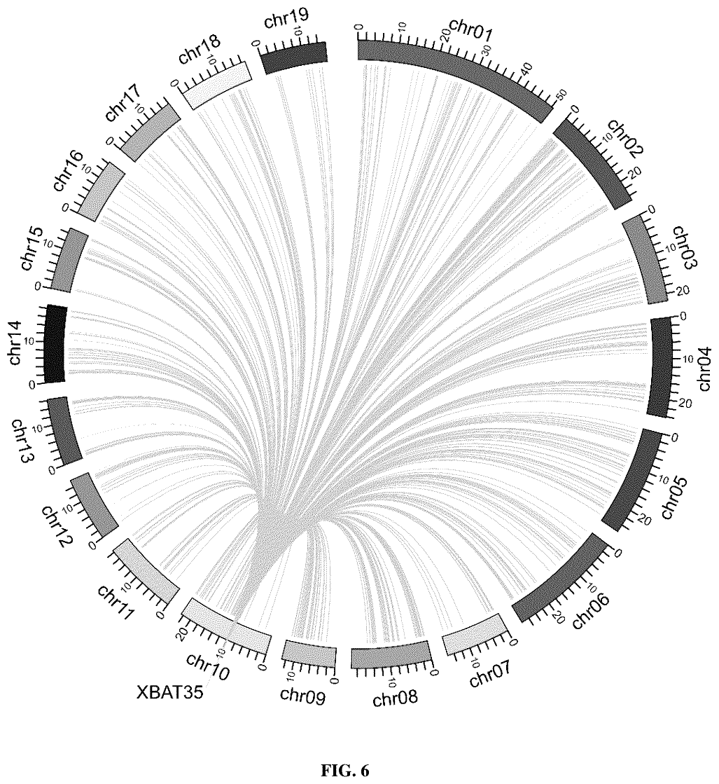

eQTL mapping was performed by correlating 390 mature leaf and 444 developing xylem transcriptomes to >8.2 million single nucleotide polymorphisms (SNPs) and small indels to identify putative cis- and trans-regulatory elements in the Populus trichocarpa GWAS mapping panel. A putative cis-regulatory element was identified that was associated with the expression of the P. trichocarpa gene model, Potri.010G070800, annotated as a PtrXB38 and was predicted to encode a C3HC4-type E3 ligase (FIGS. 1A-E). Moreover, this putative cis-element co-located with a trans-eQTL hotspot that was significantly associated with expression of more than 500 genes in developing xylem and mature leaf transcriptomes (FIG. 6). Specifically, 21 SNPs, encompassing a 20.83-kb interval, exhibited associations with expression levels of 492 genes in xylem tissue (P-values≤9.85E-11), while 10 SNPs within a 6.47-kb interval were significantly associated with expression of 81 genes in leaf tissue (P-values≤9.41E-11). PtrXB38 was the only annotated gene found in both intervals. In particular, SNP Chr10:9762348 was significantly associated with higher expression of PtrXB38 in developing xylem and leaf (FIGS. 1F, G), as well as expression levels of its putative targets described below.

Among putative targets for the PtrXB38 trans-eQTL were 73 and 18 transcription factors expressed in xylem and leaf, respectively. These included multiple members of the MYB, AP2/ERF, HSF, bHLH, GT and NAC families. Multiple transcription factor families and their putative downstream targets were previously implicated in plant organ development. Examples include genes previously implicated in callus formation and root development, such as SCZ and OBP4 (Ramirez-Parra, E. et al., New Phytologist 213, 1787-1801, 2017; Ten Hove, C. A. et al., Current Biology 20, 452-457, 2010) as well as regulators of root growth and xylem development, such as TCP20 and SVP (Zhang, J. et al., Nat Plants 5, 1033-1042, 2019; Li, C. et al., Proceedings of the National Academy of Sciences of the United States of America 102, 12978-12983, 2005) (FIGS. 1H-O). Transcript abundance of these genes was significantly associated with SNPs within the PtrXB38 locus including Chr10:9762348 mentioned above (FIGS. 1P-S). Given robust numbers of putatively co-regulated genes in xylem and leaf, a three-layer network was constructed by assessing shared targets among these transcription factors that were associated with the PtrXB38. This overlap suggested that those shared targets may be regulated by PtrXB38 via these transcription factors. This resulted in a network of putatively co-regulated gene modules falling under the overall regulation of PtrXB38 (FIG. 1T). On one hand, ARF and ERF transcription factors served as two high-rank nodes in the regulatory network, suggesting plant hormones, especially auxin and ethylene signaling pathways, may be involved in PtrXB38-regulated developmental processes (FIG. 1T). On the other hand, the MYB and bHLH were identified as direct subordinated transcription factors, suggesting that PtrXB38 may regulate MYB/bHLH-mediated biological processes, such as secondary metabolism (FIG. 1S) (Xu, W., Trends in Plant Science 20, 176-185, 2015).

### Example 3: PtrXB38 Promotes the Formation of Adventitious Roots

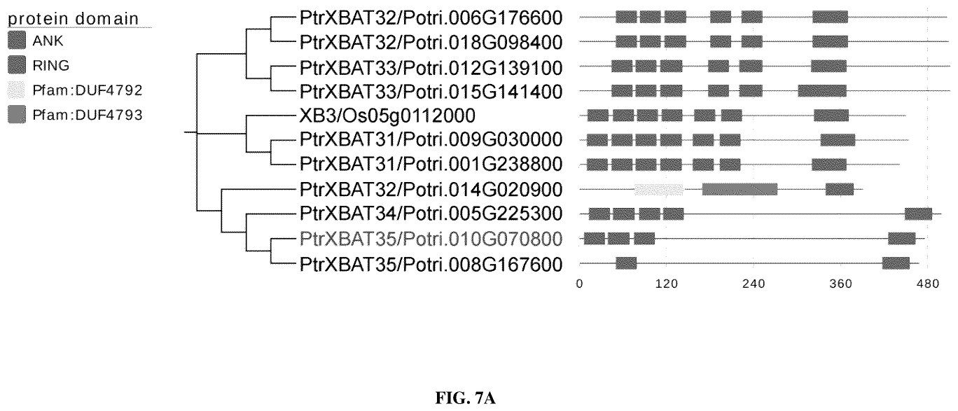

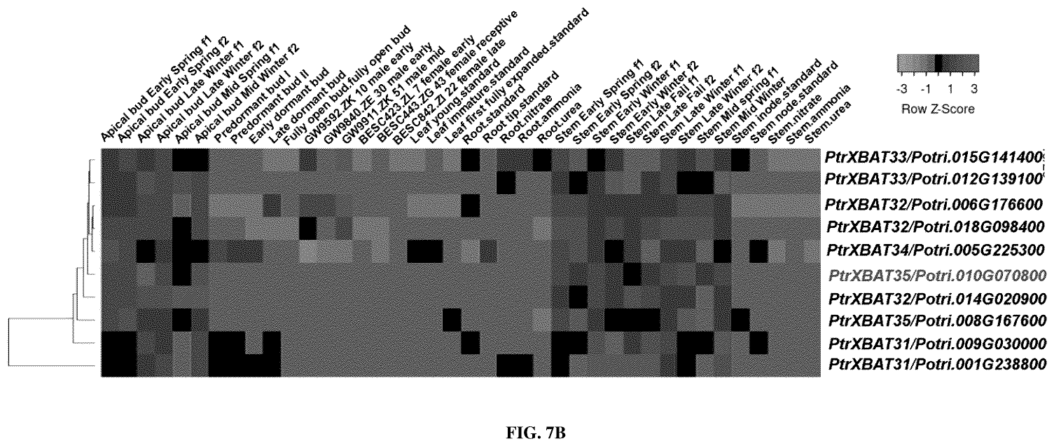

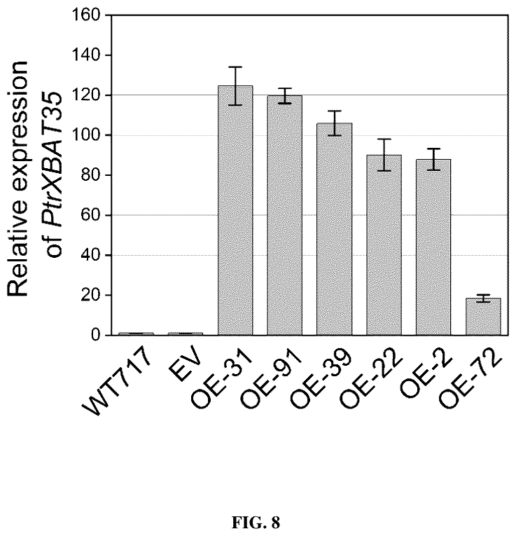

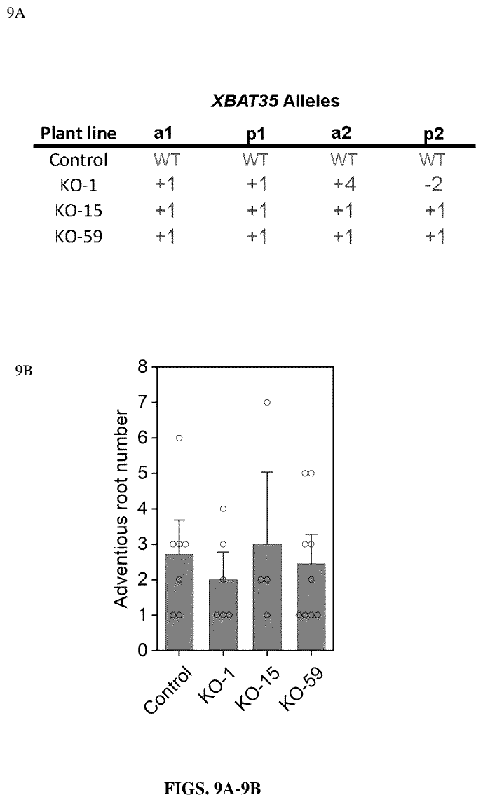

Given the number of developmental regulators implicated in the predicted PtrXB38 regulatory network, its function was assessed by generating and characterizing overexpression (OE) and knock-out (KO) transgenic plants in the P. tremula x P. alba ‘INRA 717-1B4’ background. Two independent lines, OE-72 and OE-22, in which the expression of PtrXB38 was 18- to 90-fold higher compared to empty vector control, were selected for further analyses (FIG. 8). In contrast to the control plants, PtrXB38-OE plants not only developed larger and more prolific ARs, but also produced more aerial roots (FIGS. 2A-D). When micro-propagated synchronously in ½ MS medium, PtrXB38-OE plants generated 6 to 12 ARs compared to 3 ARs in control plants. AR formation in the OE lines was also correlated with the PtrXB38 transgene expression level (FIG. 2E and FIG. 8). Alternatively, knock-out mutants of PtrXB38 did not show any difference in AR formation when compared to control (FIGS. 9A-9B). It is hypothesized that this is due to the genetic redundancy of the XBAT gene family, as there are eleven members in Populus (FIGS. 6 and 7A) and two of them have previously been reported to be involved in root development (Trupiano, D. et al., Planta 238, 271-282, 2013; Song, Y. et al., Journal of Experimental Botany, 2020).

Considering the significant differences in AR formation between OE transgenics and empty vector controls, transcriptional responses of known AR regulators using qRT-PCR analysis were assessed. As an early-stage regulator of AR formation, PtWOX5, which is expressed in the stem cell niche and is required for maintaining root meristem activity (Li, J. et al., Tree Physiology 38, 139-153, 2017), was highly up-regulated in PtrXB38-OE plants (FIG. 2F). Additionally, transcript levels of PtWOX5's upstream activator, PtSHR (Xuan, L. et al., Plant Cell, Tissue and Organ Culture (PCTOC) 117, 253-264, 2014), were also significantly increased in the PtrXB38-OE plants (FIG. 2G). Expression of PtrAIL1, another AP2/ERF family transcription factor gene responsible for root meristem and primordium formation (Rigal, A. et al., Plant Physiol 160, 1996-2006, 2012), was likewise significantly increased in PtrXB38-OE (FIG. 2J). Both WOX5 and AIL1 were reported to be involved in auxin-dependent AR development (Druege, U. et al., Frontiers in Plant Science 7, 2016). In line with this view, it was found that PtrXB38-OE resulted in up-regulation of auxin signaling and transport genes PtARF15 and PtPIN1 (FIG. 2H, I).

### Example 4: Generation of PtrXB38-OE and -KO Plants in Populus

The full-length coding sequence of PtrXB38 was PCR amplified from xylem cDNA of P. trichocarpa ‘Nisqually-1’ and assembled into binary vector p201N-Cas9 (Addgene plasmid 59175) (Jacobs, T. B. et al., BMC Biotechnol 15, 16, 2015) to replace SpCas9 behind the double 35S promoter using NEBuider HiFi DNA assembly cloning kit (New England Biolabs). The generation and genotyping of PtrXB38-KO transgenic Populus plants in Populus tremula x alba ‘IRNA 717-1B4’ followed the methods as described (Tsai, C.-J. et al., Plant Physiol 183, 123-136, 2020). To generate the PtrXB38 knockout construct, SwaI-digested p201N-Cas9 vector was PCR amplified by Q5 High-Fidelity DNA Polymerase (New England BioLabs) with primers. The MtU6.6 promoter and scaffold fragments were also PCR amplified from the pUC-gRNA shuttle vector (Addgene plasmid 47024) (Jacobs, T. B. et al., BMC Biotechnol 15, 16, 2015) using tailed primers overlapping with p201N. The p201N-Cas9 vector, U6.6 promoter, scaffold and a pair of tailed primers containing the gRNA sequence were then assembled together using NEBuilder HiFi DNA Assembly Cloning Kit (New England Biolabs). Both constructs were fully sequenced, and heat-shock transformed into Agrobacterium tumefaciens strain C58/GV3101 (pMP90). The Populus transformation and regeneration was performed as described (Tsai, C.-J. et al., Plant Physiol 183, 123-136, 2020) with the following modifications: shoot elongation media contained 0.05 mg/L 6-benzylaminopurine, all media were additionally supplemented with 200 mg/L L-glutamine and solidified with 3 g/L gellan gum, leaf discs following cocultivation were washed with 200 mg/L cefotaxime and 300 mg/L timentin, and transformants were maintained on appropriate media supplemented with 100 mg/L kanamycin, 200 mg/L cefotaxime and 300 mg/L timentin.

Mutation patterns were determined by amplicon sequencing as described (Tsai, C.-J. et al., Plant Physiol 183, 123-136, 2020). Gene-specific degenerate primers flanking both on-target (Potri.010G070800) and off-target (genome duplicate Potri.008G167600) sites were used for the first PCR, followed by PCR-barcoding, both using GoTaq G2 Green Master Mix (Promega). The pooled amplicon library was quantified by a Qubit Fluorometer (Invitrogen) prior to be sequenced on an Illumina MiSeq using a Nano PE150 flow cell at the Georgia Genomics and Bioinformatics Core, University of Georgia. After de-multiplexing, data were analyzed by the AGEseq program (Tsai, C.-J. et al., Plant Physiol 183, 123-136, 2020).

### Example 5: Involvement of Auxin and Ethylene Signals in PtrXB38-Mediated Adventitious Root Formation

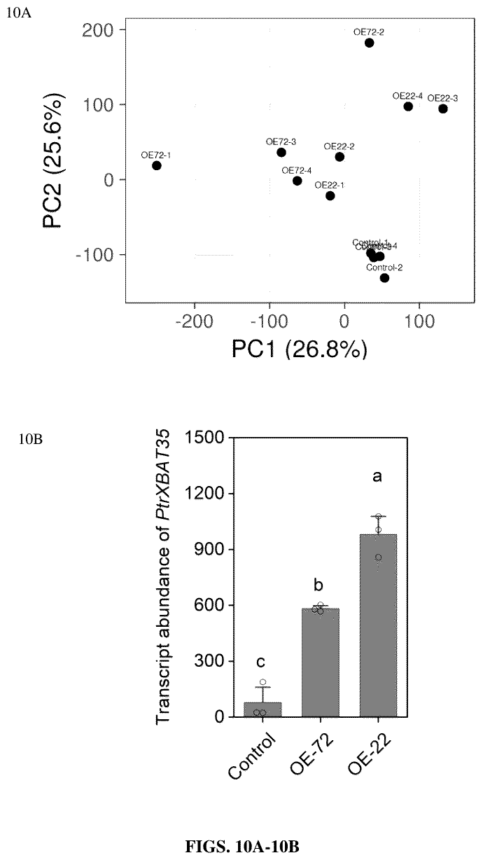

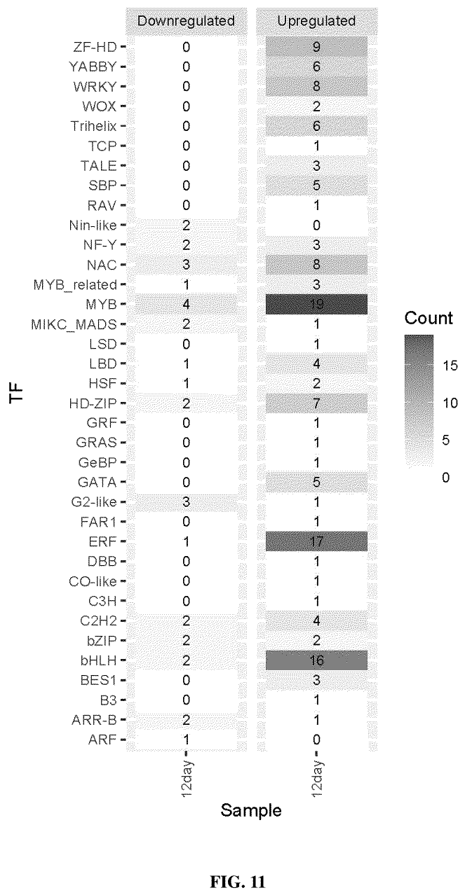

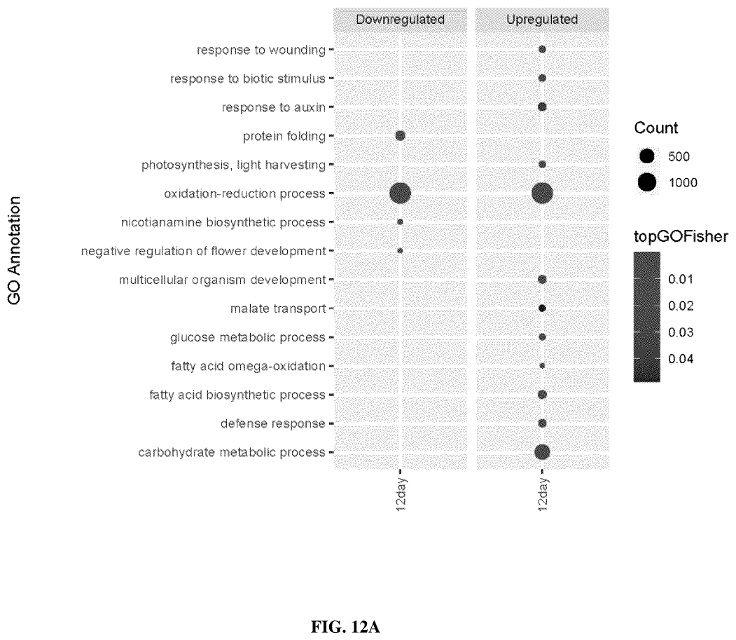

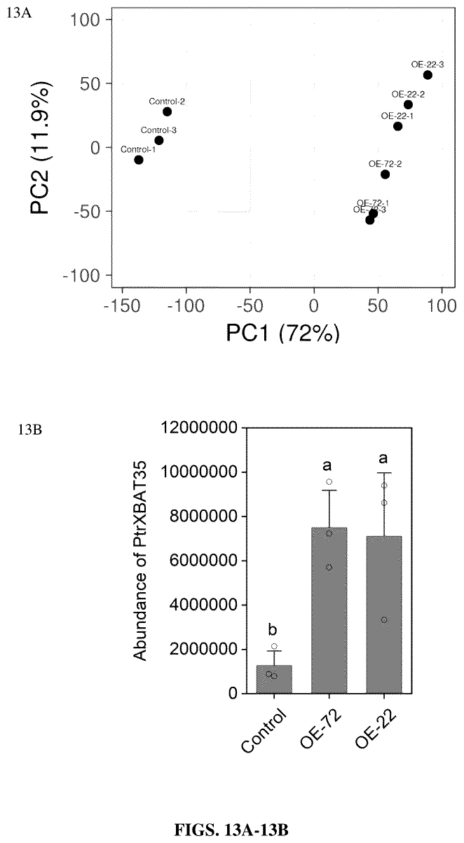

To better understand the molecular mechanism of PtrXB38 in the regulation of root development, transcriptome and proteome analyses were performed to identify changes in gene expression and protein abundance in the roots of 12-day-old PtrXB38-OE plants. Principal component analysis revealed separate clustering of the two PtrXB38-OE lines from the control group, reflecting the global transcriptomic and proteomic changes induced by over-expression of PtrXB38 (FIGS. 10A and 13A). Both transcript level and protein abundance of PtrXB38 shown by RNA-seq and proteomics results confirmed that lines OE-72 and OE-22 are bona fide PtrXB38-OE plants (FIGS. 10B and 13B). Both transcriptome and proteome analyses confirmed the upregulation of a large number of TF families, such as MYB, ERF, and bHLH, supporting the identification of PtrXB38 as a trans-eQTL hotspot (FIG. 11).

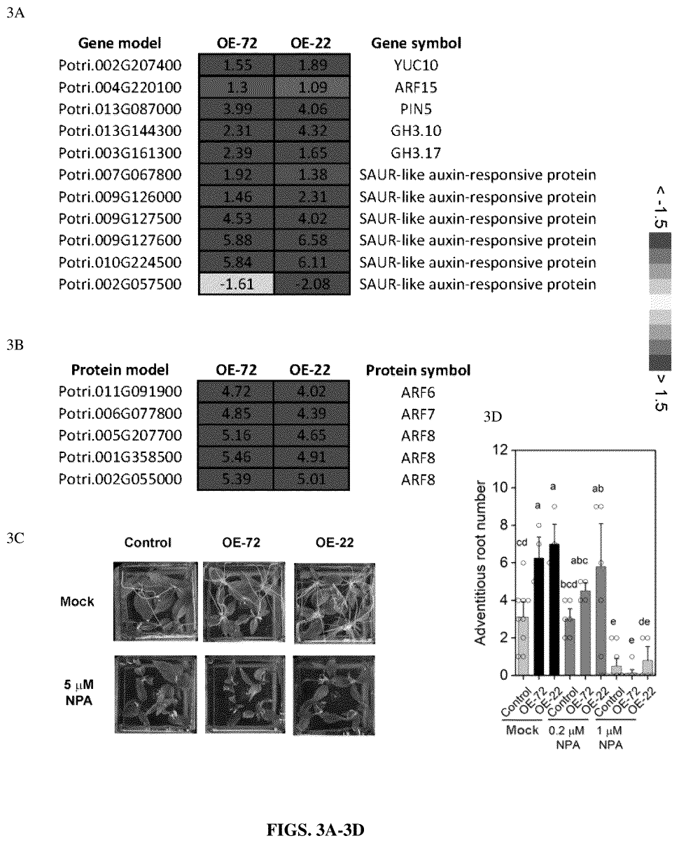

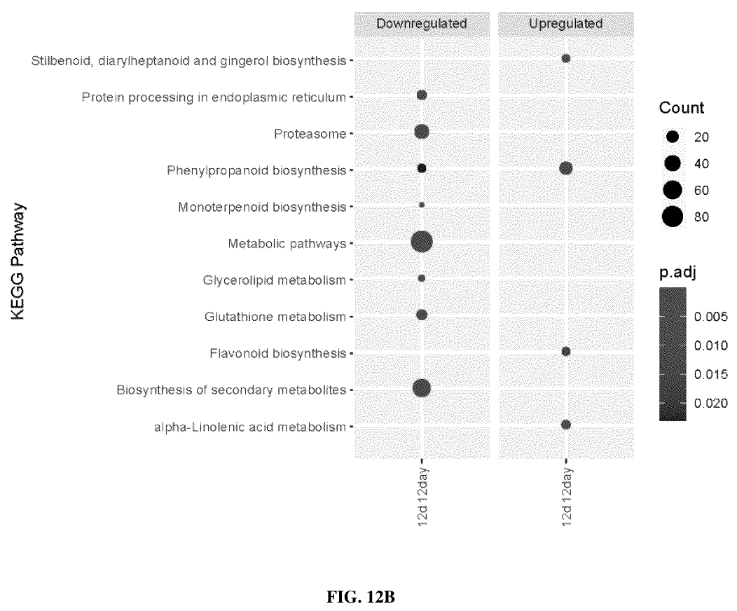

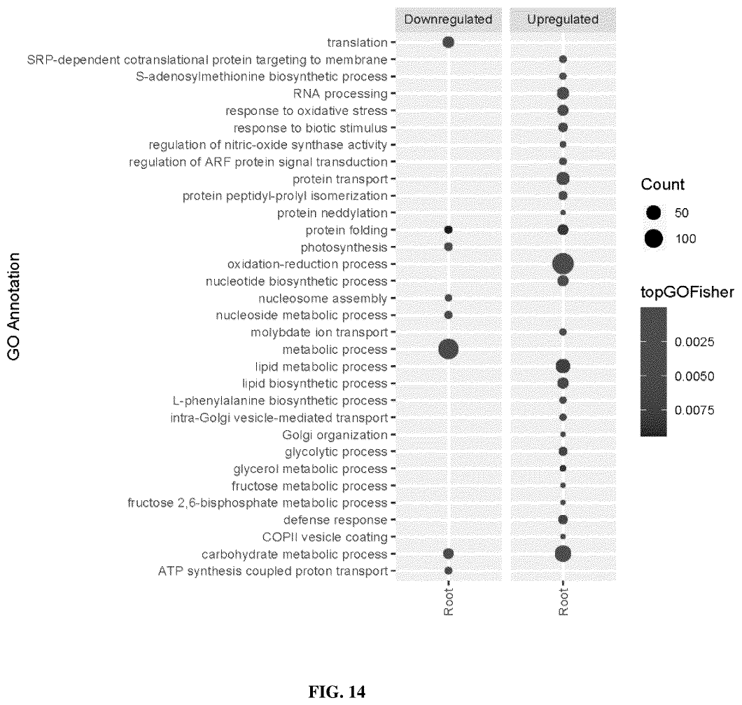

The differentially expressed genes/proteins (DEGs/DEPs, FDR≤0.05) were further analyzed by Gene Ontology (GO) and KEGG pathway enrichments. GO terms “response to wounding”, “response to auxin”, and “multicellular organism development” were overrepresented. Similarly, “regulation of ARF protein signal transduction” was also enriched among the DEPs (FIG. 14). These results are consistent with previous eQTL predictions and support the idea that PtrXB38 regulation of root development involves wounding response and auxin signaling (FIG. 12A-B). This insight is corroborated by the findings of auxin biosynthesis, signaling pathway, transport, and response related genes/proteins were highly induced in PtrXB38-OE plants from transcriptome and proteome profiling. Notably, ARF proteins were significantly accumulated in the PtrXB38-OE root samples (FIGS. 3A, B). To further verify the contribution of auxin signaling in PtrXB38-controlled root development, an auxin transport inhibitor NPA was applied during root formation. It was found that 5 μM NPA completely blocked root formation in both control and PtrXB38-OE micro-cuttings, and the promotive effect of PtrXB38 on AR formation was gradually attenuated by increasing NPA concentrations (FIGS. 3C, D). These results suggest that PtrXB38-regulated root development depends on auxin transport and signaling.

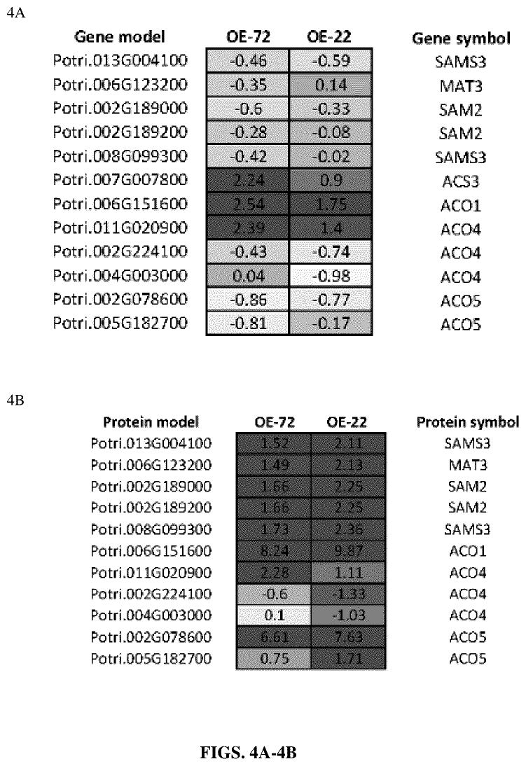

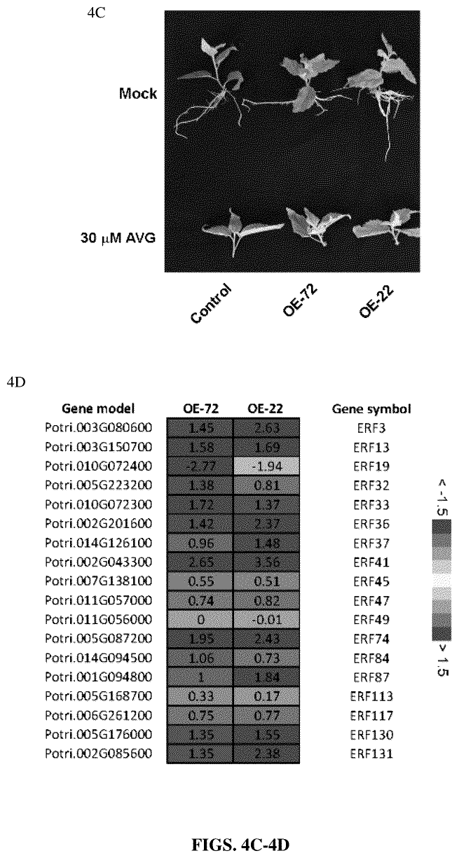

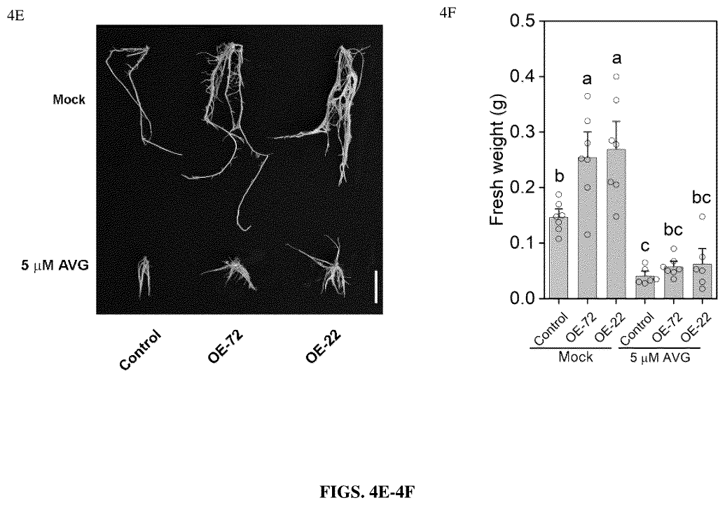

GO enrichment analysis for DEPs also identified proteins under the term “S-adenosylmethionine biosynthetic process”, indicating PtrXB38 may affect ethylene biosynthesis (FIG. 14). In fact, it has been established that the E3 ligase XBAT32 specifically mediates ACS degradation through the ubiquitin/26S proteome pathway in Arabidopsis (Druege, U. et al., Frontiers in Plant Science 7, 2016; Prasad, M. E. et al., Plant Physiol 153, 1587-96, 2010). However, a decreased abundance of ACS proteins in PtrXB38-OE root samples was not observed. Instead, it was found that several ethylene biosynthesis related enzymes, such as SAMs and ACOs, were highly abundant (FIG. 4B). In addition to an increased transcript abundance (FIG. 4A), their elevated protein abundance might be indicative of protein stability regulation. Furthermore, many ERF genes were up-regulated in PtrXB38-OE root samples (FIG. 4D). All these lines of evidence suggest that PtrXB38 also enhances ethylene biosynthesis and signaling. This inference was supported by the observation that ethylene biosynthesis inhibitor, AVG, inhibited adventitious root formation at high concentrations and suppressed AR promotion by PtrXB38-OE at lower concentrations (FIGS. 4C, E, F). Therefore, it was concluded that both auxin and ethylene signaling are required for PtrXB38-mediated AR development. It is known that auxin and ethylene regulate each other's biosynthesis and signaling (Muday, G. K. et al., Trends in Plant Science 17, 181-195, 2012; Qin, H. & Huang, R., International Journal of Molecular Sciences 19, 3656, 2018), but it remains elusive whether it is the biosynthesis and signaling of auxin or ethylene that is directly regulated by PtrXB38. Future studies on identification of PtrXB38 direct targets may provide answers to this question. Strikingly, neither XB38-OE nor xb38 null mutant in Arabidopsis showed differences in root development, possibly indicating a functional diversity of XB38 between herbaceous and woody species (Carvalho, S. D. et al., Mol Plant 5, 1295-309, 2012).

### Example 6: PtrXB38 Increases Flavonoids in Roots, Plant Defense Against Pathogens, Flooding Tolerance and Carbon Dioxide (C02) Absorption or Sequestration

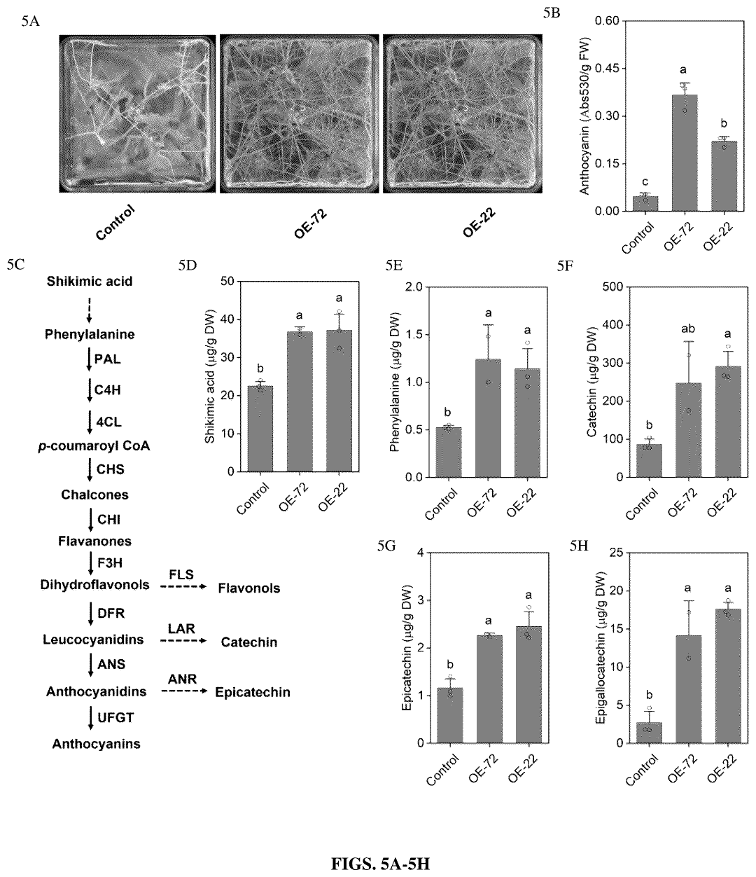

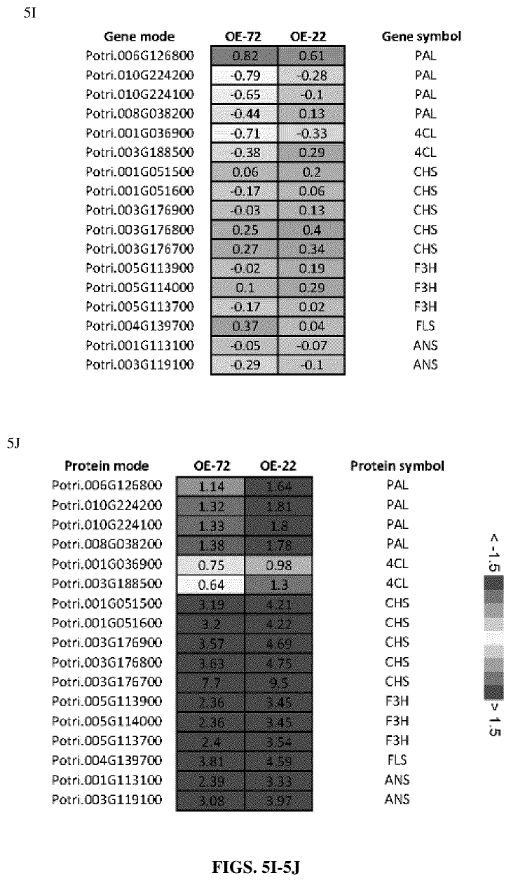

It has been suggested that ethylene biosynthesis and signaling can affect secondary metabolism in plants (Buer, C. S. et al., Plant physiology 140, 1384-1396, 2006; Ke, S. W. et al., Botanical Studies 59, 11, 2018; Ma, W. et al., Horticulture Research 8, 43, 2021). The eQTL-predicted targets of PtrXB38, such as MYB and bHLH, are known to regulate secondary metabolism pathways, such as flavonoid biosynthesis (Constabel, C. P., J Agric Food Chem 66, 9882-9888, 2018). Therefore, PtrXB38 could play a role in the regulation of secondary metabolism. Indeed, MYB123 and bHLH42 were among the DEGs identified from the root of PtrXB38-OE plants, and KEGG and MapMan analysis of the DEGs and DEPs identified “flavonoid biosynthesis” and “glycerolipid metabolism” as overrepresented (FIG. 12B). The reddish coloration of mature roots of PtrXB38-OE plants also suggested possible accumulation of photoreactive metabolites. Therefore, metabolite profiling for the roots of PtrXB38-OE plants was performed. The increased abundance of anthocyanins could partially explain the brown or red colors of the OE roots (FIGS. 5A and 5B).

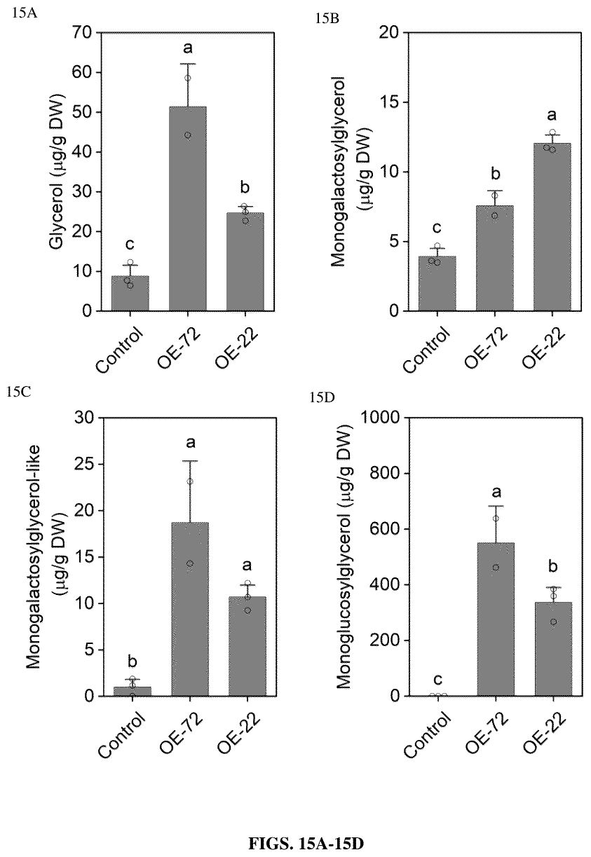

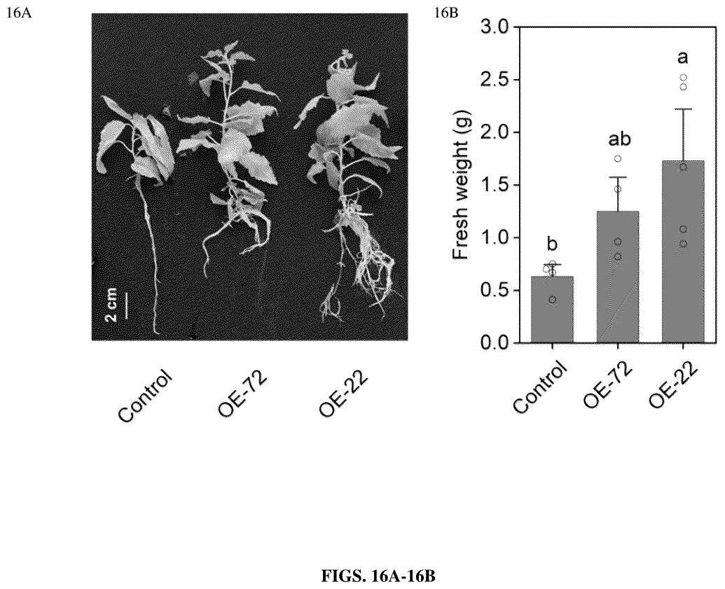

Flavan-3-ols, such as catechin and epicatechin, are crucial compounds for plants to adapt to biotic and abiotic stresses (Ullah, C. et al., New Phytologist 221, 960-975, 2019). Based on gas chromatography-mass spectrometry, three flavan-3-ols were significantly increased in the roots of PtrXB38-OE plants. Accordingly, the shikimate-phenylpropanoid pathway intermediates, shikimic acid and phenylalanine, were also significantly elevated in PtrXB38-OE roots (FIGS. 5C-5H). Although transcript elevation of phenylpropanoid and flavonoid biosynthetic genes were not observed, protein levels of PAL, 4CL, CHS, F3H, and ANS were significantly increased, indicating possible post-transcriptional regulations. Nonetheless, it was concluded that PtrXB38 affects flavonoid biosynthesis in roots. Consistent with the gene expression changes of glycerol metabolism in PtrXB38-OE plant roots, dramatic increases of glycerol, monoglucosylglycerol and monogalactosylglycerol were observed, while glycerol-1/3-phosphate content was reduced (FIG. 15A-D). The higher abundances of flavonoids and glycerolipids could increase the resilience of PtrXB38-OE plant roots to environmental stresses. In line with this view, the homologue of PtrXB38 in Arabidopsis, XB38 has been reported as an important regulator in biotic and abiotic stress response (Liu, H. et al, Plant Physiology 175, 1469-1483, 2017; Li, Q. et al., The Plant Journal 104, 1712-1723, 2020; Yu, F. et al., Molecular Plant, 2020). Taken together, root metabolic changes accompanied with root system enlargement suggest that PtrXB38 is an important regulator in root development and possibly also stress response (FIG. 16A-B).

PtrXB38-OE transgenic plant generated more adventitious and lateral roots especially fine roots in Populus. The changed root system facilitates plant nitrogen uptake, which requires more CO₂ absorption to fix the nitrogen. In addition, PtrXB38-OE transgenic poplars enhanced salicyl alcohol production, which can be served as carbon source for Populus beneficial microbial community. Enhanced root system provides more root-microbial interface. The rhizosphere microbiome in turn will promote plant nutrient and carbon uptake. Therefore, PtrXB38 can be a candidate gene to be engineered for carbon sequestration.

Recent advances reveal that phytohormone signaling contributes to shaping plant root architecture in both agronomic crops and bioenergy woody species (Csukasi, F. et al., Biotechnology Journal 4, 1293-1304, 2009; Depuydt, S. & Hardtke et al., Current Biology 21, R365-R373, 2011). Here, the present disclosure reports that a predicted E3 ligase gene, PtrXB38, identified by GWAS mapping, controls root development by regulating the biosynthesis and signaling of auxin and ethylene. The present disclosure showcases that eQTL genetic mapping, followed by functional validation, opens new prospects of discovering novel plant development regulators in woody plants (Zhang, J. et al., New Phytologist 220, 502-516, 2018; Labbé, J. et al., Nature Plants 5, 676-680, 2019; Muchero, W. et al., Proceedings of the National Academy of Sciences 115, 11573-11578, 2018).

### Example 7: Materials and Methods

Plant Growth Conditions: The tissue culture plants were maintained in ½ MS media (½ MS basal salts, 20 g/L sucrose, 1× Gamborg's B5 vitamin mixture, 0.3% Gelzan) supplemented with 0.1 mg/L IBA and grown in growth chambers (16 h:8 h, light:dark) with white fluorescent light (100 μmol m⁻² s⁻¹) at 22° C. To assess adventitious root development phenotype, 2-3 cm length of shoot tops from 1-month-old tissue culture saplings were excised and placed in fresh medium for root induction. Root number was counted 12 days after subculture. For chemical treatment, filter-sterilized auxin transport inhibitor naphthylphthalamic acid (NPA) and ethylene biosynthesis inhibitor aminoethoxyvinylglycine (AVG) were added in the ½ MS medium.

RNA Extraction and Quantitative RT-PCR Analysis: Total RNA was isolated from 7-day-old root samples using a Sigma plant total RNA kit according to the manufacturer's instructions. Reverse transcriptional reactions were performed using SuperScript III kit (Invitrogen) according to the manufacturer's instructions. Quantitative PCR was performed using a StepOne real-time PCR system (Thermo Fisher Scientific) and Maxima SYBR Green/ROX qPCR master mix (Thermo Fisher Scientific). PtEF1B was used as a reference gene in qRT-PCR and the gene expression level was calculated by the 2^{-ΔΔCT} method.

Transcriptome Analysis: Total RNA was isolated from the 12-day-old root samples using the Sigma plant total RNA kit according to the manufacturer's instructions. Library construction and sequencing were performed using the DNBseq platform at BGI. The raw reads have been deposited at the SRA and can be accessed with the BioProject ID PRJNA785328. Clean reads were mapped to the P. tremula×P. alba clone INRA 717-1B4_v1.1 reference genome with STAR v2.6.1b (Dobin, A. et al., Bioinformatics 29, 15-21, 2013). FeatureCounts 1.6.3 in unstranded mode was used to generate raw gene counts, excluding multimapping reads (Zhou, X. & Stephens, M., Nat Genet 44, 821-4, 2012). The primary isoform of PtrXBAT35 (Potri.010G070800.6) in P. trichocarpa was manually curated to correct exon-intron prediction in the 717-1B4 v1.1 annotation. FPKM normalized counts were generated with RSEM, default parameters were used. DESeq2 v1.22.2 was used to identify differentially expressed genes (Love, M. I. et al., Genome Biol 15, 550, 2014). TopGO was used to generate gene ontology enrichments and KOBAS was used to generate KEGG enrichments (Alexa, A. et al., Bioinformatics 22, 1600-7, 2006; Xie, C. et al., Nucleic Acids Res 39, W316-22, 2011). Before visualization of pathways with MapMan 3.5.1.R2, Mercator4 v2.0 was used to annotate the 717-1B4 v1.1 proteome (Schwacke, R. et al., Mol Plant 12, 879-892, 2019).

Proteomics Analysis: The Protein identification and quantification of the poplar root samples were described previously (Wang, J. et al., Comput Struct Biotechnol J 19, 1917-1927, 2021) with minor modifications. Briefly, liquid nitrogen frozen and ground root tissue samples were solubilized in 1 mL lysis buffer (4% sodium dodecyl sulfate (SDS) with 10 mM dithiothreitol (DTT) in 100 mM ammonium bicarbonate (ABC)). Tissue samples were boiled in a heat-block for 5 min at 90° C., vortexed and boiled for an additional 5 min at 90° C. Samples were centrifuged at max speed for 5 min and supernatants were collected. Samples were then alkylated by incubating with 30 mM iodoacetamide for 15 min in dark to prevent reformation of disulfide bonds. Proteins were then extracted using a chloroform-methanol extraction protocol using methanol, chloroform, and LC/MS water in the ratio of 4:1:3. The protein layer was separated and washed using 100% methanol. Protein layer was air-dried, and this dried pellet was reconstituted in 3 mL sodium deoxycholate (SDC) solution made up of 1% SDC in 100 mM ABC. Protein concentration was measured using nanodrop (Thermo Scientific).

Each sample was adjusted to be 2 mg of total protein. Proteins were digested with two separate and sequential aliquots of sequencing grade trypsin (Promega) of 1:50 (wt/wt) protein:trypsin ratio. Samples were first digested for 3 hours, followed by overnight digestion. After digestion, SDC was removed by precipitating with 1% formic acid followed by ethyl acetate wash. Samples were then lyophilized in a SpeedVac Concentrator (Thermo Fischer Scientific). Peptide samples were desalted on Pierce peptide desalting spin column (Thermo Scientific) as per the manufacturer's instructions. After speed-vac concentration, dry samples were suspended in 500 mL of 0.1% formic acid solution. Peptide concentrations were then measured using nanodrop and 20 μg of each sample was aliquoted in autosampler vial for bottom-up proteomics.

All samples were analyzed using two-dimensional (2D) liquid chromatography (LC) on an Ultimate 3000 RSLCnano system (Thermo Fisher Scientific, Waltham, MA) coupled with a Q Exactive Plus mass spectrometer (Thermo Fisher Scientific, Waltham, MA) as previously described⁴⁹. In brief, peptides were separated on an in-house built strong cation exchange (SCX) Luna trap column (5 μm, 150 μm×50 mm; Phenomenex, Torrance, CA) followed by a nanoEase symmetry reversed-phase (RP) C18 trap column (5 μm, 300 μm×50 mm; Waters, Milford, MA) and washed with an aqueous solvent. Proteomics samples were separated and analyzed across three successive SCX fractions of increasing concentrations of ammonium acetate (35 mM, 50 mM, and 500 mM). These fractions were followed by a 100-minute organic gradient (25 nL/min flow rate) to separate peptides across an in-house pulled nanospray emitter analytical column (75 μm×350 mm) packed with C18 Kinetex RP C18 resin (1.7 μm; Phenomenex, Torrance, CA). All MS data were acquired with Thermo Xcalibur (v4.2.47) using the topN method where N could be up to 10.

All MS/MS spectra collected were processed in Proteome Discoverer v2.5 (Thermo Scientific) using SEQUEST HT and Percolator. Spectral data were searched against the Populus 717 reference proteome database to which common laboratory contaminants were appended. The following parameters were set up in SEQUEST HT to derive fully tryptic peptides: MS1 tolerance=10 ppm; MS2 tolerance=0.02 Da; missed cleavages=2; Carbamidomethyl (C, +57.021 Da) as static modification; and oxidation (M, +15.995 Da) as dynamic modifications. The percolator FDR threshold was set to 1% at the PSM and peptide levels. FDR-controlled peptides were then quantified according to the chromatographic area-under-the-curve and mapped to their respective proteins. Areas were summed to estimate protein-level abundance.

For qualitative and quantitative analysis of proteome, the protein table was exported from Proteome Discoverer. For qualitative analysis, proteins were filtered to contain high false discovery (<1%) confidence and have at least 2 peptide evidence. For quantitative analysis, proteins were Log₂ transformed, LOESS normalized between the biological replicates and mean-centered across all the conditions using InfernoRDN software. Data matrix was then filtered to remove stochastic sampling; all proteins present in ⅔^{rd} of biological replicates in any condition were considered valid for quantitative analysis. Missing data were imputed by random numbers drawn from a normal distribution (width=0.3 and downshift=2.8 using Perseus software. Analysis of variance (ANOVA) followed by Tukey's HSD test was performed to identify the significantly changing proteins.

Gas Chromatography-Mass Spectrometry (GC-MS) Metabolite Profiling: Metabolites were extracted from ˜150 mg of frozen root powder twice overnight with 2.5 ml of 80% ethanol. Sorbitol (75 μl of 1 mg/ml aqueous solution) was added to the first extract as an internal standard. The two extracts were combined, and a 1 ml aliquot was dried under nitrogen. The dried extracts were silylated to produce trimethysilyl (TMS) derivatives by dissolving in 500 μl of silylation grade acetonitrile (Thermo Scientific, TS20062), followed by addition of 500 μl of N-methyl-N-trimethylsilyltrifluoroacetamide (MSTFA) with 1% trimethylchlorosilane (TMCS) (Thermo Scientific, TS48915) and heated for 1 h at 70° C. After 2 days, 1 μl was injected into an Agilent Technologies 7890A GC coupled to a 5975C inert XL MS configured as previously described (Tschaplinski, T. J. et al., Biotechnol Biofuels 5, 71, 2012), except the gas (He) flow was 1.20 ml per minute. Metabolites were identified using a Wiley Registry 10^{th} Edition combined with NIST 2014 in addition to a large custom database of TMS-derivatized compounds (Tschaplinski, T. J. et al., Biotechnol Biofuels 5, 71, 2012). Metabolite peaks were extracted using key mass-to-charge (m/z) selected ions to minimize interference with co-eluting metabolites and quantified as previously described (Tschaplinski, T. J. et al., Biotechnol Biofuels 5, 71, 2012) scaling back to the total ion chromatogram and normalizing to internal standard recovered, volume analyzed, and mass extracted.

What is claimed is:

1. A genetically modified plant, plant cell or plant tissue, comprising an exogenous nucleic acid which comprises a nucleotide sequence encoding a PtrXB38 polypeptide or a homolog thereof, wherein the genetically modified plant, plant cell or plant tissue displays an enhanced expression of the PtrXB38 polypeptide or the homolog thereof and an increase in root development compared to a wild-type plant, plant cell, or plant tissue.

2. The genetically modified plant, plant cell or plant tissue of claim 1, wherein the nucleotide sequence has at least 90% sequence identity to the nucleotide sequence shown in SEQ ID NO: 1.

3. The genetically modified plant, plant cell or plant tissue of claim 1, wherein the PtrXB38 polypeptide or the homolog thereof has at least 90% sequence identity to the amino acid sequence shown in SEQ ID NO: 2.

4. The genetically modified plant, plant cell or plant tissue of claim 1, wherein the exogenous nucleic acid is stably integrated into the plant genome.

5. The genetically modified plant, plant cell or plant tissue of claim 1, wherein the nucleotide sequence is operably linked to a heterologous promoter.

6. The genetically modified plant, plant cell or plant tissue of claim 5, wherein the heterologous promoter is a constitutive promoter, an inducible promoter, or a native promoter.

7. The genetically modified plant, plant cell or plant tissue of claim 5, wherein the heterologous promoter is a 35S promoter.

8. The genetically modified plant, plant cell or plant tissue of claim 1, wherein the plant is a monocot or a dicot.

9. The genetically modified plant, plant cell or plant tissue of claim 8, wherein the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

10. The genetically modified plant, plant cell or plant tissue of claim 9, wherein the plant is Populus.

11. The genetically modified plant, plant cell or plant tissue of claim 10, wherein the plant is Populus trichocarpa.

12. The genetically modified plant, plant cell or plant tissue of claim 1, wherein the root development comprises adventitious root formation.

13. The genetically modified plant, plant cell or plant tissue of claim 12, wherein the adventitious root formation requires auxin transport and signaling.

14. The genetically modified plant, plant cell or plant tissue of claim 12, wherein the adventitious root formation requires ethylene biosynthesis and signaling.

15. The genetically modified plant, plant cell or plant tissue of claim 1, wherein the enhanced expression of the PtrXB38 polypeptide or the homolog thereof increases flavonoids in roots, thereby increasing the resilience of the plant roots to environmental stresses.

16. The genetically modified plant, plant cell or plant tissue of claim 1, wherein the plant displays one or more of the following characteristics:
has improved plant defense against pathogens by increasing production of ethylene and jasmonates compared to a wild type plant;
has increased flooding tolerance compared to a wild type plant; and
has increased carbon dioxide (CO₂) absorption or carbon dioxide (CO₂) sequestration compared to a wild type plant.

17. A method of increasing root development in a plant, plant cell or plant tissue, comprising introducing into the plant, plant cell or plant tissue, an exogenous nucleic acid sequence encoding a PtrXB38 polypeptide or a homolog thereof, thereby obtaining a genetically modified plant, plant cell or plant tissue, wherein the genetically modified plant, plant cell or plant tissue displays an enhanced expression of the PtrXB38 polypeptide or the homolog thereof and an increase in root development compared to a wild-type plant, plant cell, or plant tissue.

18. The method of claim 17, wherein the nucleotide sequence has at least 90% sequence identity to the nucleotide sequence shown in SEQ ID NO: 1.

19. The method of claim 17, wherein the PtrXB38 polypeptide or the homolog thereof has at least 90% sequence identity to the amino acid sequence shown in SEQ ID NO: 2.

20. The method of claim 17, wherein the exogenous nucleic acid is stably integrated into the plant genome.

21. The method of claim 17, wherein the nucleotide sequence is operably linked to a heterologous promoter.

22. The method of claim 21, wherein the heterologous promoter is a constitutive promoter, an inducible promoter, or a native promoter.

23. The method of claim 21, wherein the heterologous promoter is a 35S promoter.

24. The method of claim 17, wherein the plant is a monocot or a dicot.

25. The method of claim 24, wherein the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

26. The method of claim 25, wherein the plant is Populus.

27. The method of claim 26, wherein the plant is Populus trichocarpa.

28. The method of claim 17, wherein the root development comprises adventitious root formation.

29. The method of claim 28, wherein the adventitious root formation requires auxin transport and signaling.

30. The method of claim 28, wherein the adventitious root formation requires ethylene biosynthesis and signaling.

31. The method of claim 28, wherein the enhanced expression of the PtrXB38 polypeptide or the homolog thereof increases flavonoids in roots, thereby increasing the resilience of the plant roots to environmental stresses.

32. A method of improving plant defense against pathogens in a plant, plant cell or plant tissue comprising enhancing expression of a PtrXB38 polypeptide or a homolog thereof in the plant, plant cell or plant tissue, thereby increasing production of ethylene and jasmonates compared to a wild type plant.

33. The method of claim 32, wherein the nucleotide sequence has at least 90% sequence identity to the nucleotide sequence shown in SEQ ID NO: 1.

34. The method of claim 32, wherein the PtrXB38 polypeptide or the homolog thereof has at least 90% sequence identity to the amino acid sequence shown in SEQ ID NO: 2.

35. The method of claim 32, wherein the exogenous nucleic acid is stably integrated into the plant genome.

36. The method of claim 32, wherein the nucleotide sequence is operably linked to a heterologous promoter.

37. The method of claim 36, wherein the heterologous promoter is a constitutive promoter, an inducible promoter, or a native promoter.

38. The method of claim 36, wherein the heterologous promoter is a 35S promoter.

39. The method of claim 32, wherein the plant is a monocot or a dicot.

40. The method of claim 39, wherein the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

41. The method of claim 40, wherein the plant is Populus.

42. The method of claim 41, wherein the plant is Populus trichocarpa.

43. A method of increasing flooding tolerance in a plant, plant cell or plant tissue comprising enhancing expression of a PtrXB38 polypeptide or a homolog thereof in the plant, plant cell or plant tissue, thereby increasing root development compared to a wild type plant.

44. The method of claim 43, wherein the nucleotide sequence has at least 90% sequence identity to the nucleotide sequence shown in SEQ ID NO: 1.

45. The method of claim 43, wherein the PtrXB38 polypeptide or the homolog thereof has at least 90% sequence identity to the amino acid sequence shown in SEQ ID NO: 2.

46. The method of claim 43, wherein the exogenous nucleic acid is stably integrated into the plant genome.

47. The method of claim 43, wherein the nucleotide sequence is operably linked to a heterologous promoter.

48. The method of claim 47, wherein the heterologous promoter is a constitutive promoter, an inducible promoter, or a native promoter.

49. The method of claim 47, wherein the heterologous promoter is a 35S promoter.

50. The method of claim 43, wherein the plant is a monocot or a dicot.

51. The method of claim 50, wherein the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

52. The method of claim 51, wherein the plant is Populus.

53. The method of claim 52, wherein the plant is Populus trichocarpa.

54. A method of increasing carbon dioxide (CO₂) absorption or carbon dioxide (CO₂) sequestration in a plant, plant cell or plant tissue comprising enhancing expression of a PtrXB38 polypeptide or a homolog thereof in the plant, plant cell or plant tissue, thereby increasing root development compared to a wild type plant.

55. The method of claim 54, wherein the nucleotide sequence has at least 90% sequence identity to the nucleotide sequence shown in SEQ ID NO: 1.

56. The method of claim 54, wherein the PtrXB38 polypeptide or the homolog thereof has at least 90% sequence identity to the amino acid sequence shown in SEQ ID NO: 2.

57. The method of claim 54, wherein the exogenous nucleic acid is stably integrated into the plant genome.

58. The method of claim 54, wherein the nucleotide sequence is operably linked to a heterologous promoter.

59. The method of claim 58, wherein the heterologous promoter is a constitutive promoter, an inducible promoter, or a native promoter.

60. The method of claim 58, wherein the heterologous promoter is a 35S promoter.

61. The method of claim 54, wherein the plant is a monocot or a dicot.

62. The method of claim 61, wherein the plant is selected from the group consisting of genera Acer, Afzelia, Allium, Arabidopsis, Agrostis, Avena, Betula, Brassica, Capsicum, Citrullus, Cucumis, Eucalyptus, Fagus, Festuca, Fraxinus, Fragaria, Glycine, Gossypium, Hordeum, Ipomoea, Jatropha, Juglans, Lemna, Lolium, Malus, Manihot, Medicago, Micropus, Milium, Miscanthus, Nicotiana, Oryza, Pennisetum, Phalaris, Phleum, Picea, Pinus, Poa, Populus, Prunus, Quercus, Rosa, Salix, Solanum, Sorghum, Spinacia, Tectona, Trifolium, Triticum, Panicum, Saccharum, Setaria, Zea, and Zoysia.

63. The method of claim 62, wherein the plant is Populus.

64. The method of claim 63, wherein the plant is Populus trichocarpa.


SEQUENCE LISTING (11 sequences)

SEQ ID NO: 1 - DNA, 1431 bp, Populus trichocarpa
  source 1..1431: mol_type = other DNA
atggggcaacagcaatcaaagcacgagttgctgtatcagcaagtgaaaaacggcaacatagaaggaatcaaaaaactttgcagagaaggcgctcgcctcgagtggattgataaagaaggaaaaacaccactgatattggcgtgtttggatcctcaactctttaacgtggctaaaactttgattgagctcggtgctgatgttaatgcctatcgcccaggtcgaaagggggggaatcccttgcatcatgcggcaaaaaaaggtcttgaaaatactgtcaagttacttctttcacatggagcaaatgcattaatgacgaatgatgattgtcaaactcctctagaagttgctagggcaaaagggtacggcaatgttgtccgggcaattgagagtcatatttgcctattctctggttggttgcgggagttttatggacctggatttctagaagtattggcacctcggttggtttcaagaaacatatgggctgttgttttacctactggttcccgaaatcctggaatgccttgcaagttcgaacttgccatttattctagtttgaaggatgctaaaccacgcacgataattgcattgtggaaagcaaatctggaagaaccgaagtttcaccgctccgatcatactgtgatgattgctgacaactccactaaaacacgcattaaactcgctcctgcaaatgaaagagacaagcaacagcttcagtggttttgtgatgcatgcaaaggaataccacaggtaatgcatcctctagaatttctgtccagatctcagaatctggttgttcaagcaactgctccaccatcggatgaagatccagagatagcaatggcagtaaatgcctccattcagtctgccatggcagagcaacctatttttgatacccactctagcactggagcaagttcatcctctagctggaattgccctgttaatgatggcggtcaaggtgctatggatgtgccagcaacacctcctccaaaaacaactagcagcggatgggcaccttatgaaagtgtatctagtggcagctcaactcagcaaaccaagatcttgaatagcagcgttgctgatgtccagacagcaactgatgcccaagattcagtcccatcagctccacccattgtagatgagcttatagaagatggtcccattcactatccatcaattgattccagcccccttgatttttcttccctgccccttgagaatttaccagaaaacacgggtgaaaagaaagaagatggaggttcatcatcatgtgtaatatgtttagatgctccagttgagggagcttgcattccatgtggccacatggttggatgtatgtcttgtttgaaggaaatcaaagccaaggaatggggttgccctgtgtgtcgtgccacaattaaccaggttgtaaggctctatgctgtttga

SEQ ID NO: 2 - protein, 476 aa, Populus trichocarpa
MGQQQSKHELLYQQVKNGNIEGIKKLCREGARLEWIDKEGKTPLILACLDPQLFNVAKTLIELGADVNAYRPGRKGGNPLHHAAKKGLENTVKLLLSHGANALMTNDDCQTPLEVARAKGYGNVVRAIESHICLFSGWLREFYGPGFLEVLAPRLVSRNIWAVVLPTGSRNPGMPCKFELAIYSSLKDAKPRTIIALWKANLEEPKFHRSDHTVMIADNSTKTRIKLAPANERDKQQLQWFCDACKGIPQVMHPLEFLSRSQNLVVQATAPPSDEDPEIAMAVNASIQSAMAEQPIFDTHSSTGASSSSSWNCPVNDGGQGAMDVPATPPPKTTSSGWAPYESVSSGSSTQQTKILNSSVADVQTATDAQDSVPSAPPIVDELIEDGPIHYPSIDSSPLDFSSLPLENLPENTGEKKEDGGSSSCVICLDAPVEGACIPCGHMVGCMSCLKEIKAKEWGCPVCRATINQVVRLYAV

SEQ ID NO: 3 - DNA, 7872 bp, Populus trichocarpa
  source 1..7872: mol_type = other DNA
acacaaaacaaatgacttaggatgcataaatatttccatgccatgatatactgtatgtattagataattacaaactagtagaagagtatttctgagttccagccttgatcaagttaaacaagaaaatcattcgtcaagaggcatattatttccaagagttcttttccatgcgcatgtctgggaagaagaaggtggtgttctgctcattgatcccgtttgaagcaaaggaaaggcagggggggaatattatttaaatttgcccccatgcaggatcgaactacagaccttcagtttacaagactgccgctctaccactcttgctctctcaaaagtcaatttcgttattttatgtatgatgtcctttcgggtatctgatgaggtattatcatgttcagaatttttatagttgagataagtgaattttatgtagctttcccggaaagtaccgctccgatttacctctaaataatacgagaagagaagagaaggcaatccaggaaggaaggaatccttcatttttgtttgttttgtgatacaaaaagaatggggcaacagcaatcaaagcacgagttgctgtatcagcaagtgaaaaacggcaacatagaaggaatcaaaaaactttgcagagaaggcgctcgcctcgaggttccgattattccaacttttttatctctccttttcattattttttttaataaatctaattggttttgttgtgcagtggattgataaagaaggaaaaacaccactgatattggcgtgtttggatcctcaactctttaacgtggctaaaactttgattgagctcggtgctgatgttaatgcctatcgcccaggtttccctttctttactaatttaaatattcatttgctttcttaatttttcaattaagagaaattagttcgttcttaattagttcagtactccactgcataacaaaacaggttcattttcgaatatattacggtaaaggatttttttttttttgaaggaattgcctctgcccacggattcttgattaacgtgttaattctgtttatgagtataattatttttattttttgtatcgagctgtaggtcgaaagggggggaatcccttgcatcatgcggcaaaaaaaggtcttgaaaatactgtcaagttacttctttcacatggaggtgaggcgagctcctgtacttaaagagattagattggttgtagttattttttatttaaaaatttattaaaataattatttttgatatcaatatattaaaataatttaaaaacactaaaaatatattaattaaaaaaaattaaaaatgtttcaaatttttttaaaaaaggttttgaaatgtaaaaacaaacagataattgcttttaaaagtgattttttcttgaaaattcattaaaacaccaaaaaaaattaatttgaagcaaataaaaatttaaattttttctaaaacaattttaaaacgcaaaaataaataagtaatagttgtttttcaaaatgtttttcactcgaaaattcataaaaataatatttgtttaaaaaaaatatttttgatatcagtgcatcaaaatgatttcaaaacaccaaaaaaatattaatttgaagcaaaaaaataaaaaaaaattaaattttttaaaacaaaaaacaaacaggtaatagtttctttttaaaatgttttttatttgaaaattcatcaaaataatatatgtttttagttttaaaaaatttatttttgacatcagcgcatcaaaatgatttaaaaatactaaaaaaatattaatttaaagaaaataaaaaaaattaaatttttaaaaaaatatttttaaaatacaaaaaagaatagttaatttttgcagctgtgtgtgtgtctatatatatatatatatatatatatatatattggagtgtcagcgatagtttaataagtatttgtttctttttaattttcagcaaatgcattaatgacgaatgatgattgtcaaactcctctagaagttgctagggcaaaagggtacggcaatgttgtccgggcaattgaggtatgcttaaccgttcatgctgtgatgttgtaactactttgtaactgggcaacgtgcgaaaataatttctgttaattaccatgtaaaaaagattttttcccctaattctttgcagagtcatatttgcctattctctggttggttgcgggagttttatggacctggatttctagaagtattggcacctcggttggtttcaagaaacatgtgggtatttgttgttttttaatactactttgtaagttatggaatatgttatctaggcagtttgaggttgaaaatataattacggttattttttaaaatgttttttatttaaaaatatattaaaataatattttttttattttttaaaaattatttttgatattagcgcattaaaataatttaaaaatatcaaaaaatattaatttgaagaaaaaaataaaaaaaattcaaacccttttaaaacgcaaaaacaaatataaccttagattgaaatggcttgttgcatgtaacatttgctatacagctaaatattgttctcaaaaagcactggatttgttatgcctaatagtgtcacggcttatgcagatgggctgttgttttacctactggttcccgaaatcctggaatgccttgcaagttcgaacttgccatttattctagtttgaaggtacgccaagtaaaattttctaatttgcaagaaattggataccagattcacattcattgttaaaattttataacgatgcaggcttttgtttttgtgctacctttgaaagttattttttggttctaggctctgctgtaatgatacttgtatttgtctttaagcttttggaattcttgtccggcacattcctggtttgcatcggtggtttatctttattcttgtttatattatacatagttattagatatcctacatgaggattgaaccggacaagaagccgggtttagggttatataaattgactcggatcaaccaaaattaactcggaaaaattaaaaaaaatattataagttctaatatttcatataaaaaaattaaaaaataatccatgtgaatatatgttatacttgttataaataatgaagtttaaaagattatttcaaaaggttttttatcgcatattgaaaagatattatgtgttatccttttaagttgaagtatgtaaaccaaaaagactttttatcccacattgaaaaaacataatttttttcttgtgaacataaaatatatatactaaagggcttcaaatcccacataaaaaaataaaatattattctagtatttatatagtgaactttgaaaaaagttaataaccaactaaaaaaaaaacggatctcaatcgggttttgcctggtcaaccaagtttttgctcatcccattctttgtcctcacccggaccgatctagccatcgggaaaccatgctggttcattccgggtttaataactatggtttatacacattggttggttttctagcagtgttatgggtttttaccaaaaacacacttttattataaaatttatctggttctggattcataatggttgtagctatacctgttaaccttccatgtatttaatcgatagaatgtggtgggaaacaataaatgttgtttggctctacaggtggaaatatataaaagctcaggttgagactcgaactaatggaatatatccctcttaaaaccccgtgtgagtgcctctacatgtgagtgagacacaaggttattgttcctagctaggttgaagtctacctcatcccacctagcacagtggcttgaatcaaagatattttattaagtaatccaagcacctacagtactgttacaagctgctttagtggtaaaaaaatgctgctattttgctccttctagctgttttcatggtgttgaaatagtcttcaaagatgtcaatacttcttggcattcagaaatgagaacatcgcagcgtgggaggtggtgatgtgcttagctactacttccacatccaagcatgctttgcatcaattaccacttgaacattttagtgctagaattttgcagaataggctgcattttggaacctctcactgtgtgctgcatgttaaaacaaagcaaactttgtttatgaagttgcatgaaatgggactgacgactcactttcaacggtctctttctttgatgatagatgttatcagggttttatttcttagtttagttgtccttattccttgtttgaatttgtattccaagtagtgtttacaaatgtaagatgttgtatccctatgttttaaagtgctatattttcttctcttttaagttatgactttctggttcttggtaattttcttacatattaatggacgaccaggatgctaaaccacgcacgataattgcattgtggaaagcaaatctggaagaaccgaagtttcaccgctccgatcatactgtgatgattgctgacaactccactagtaatttctcaatgatgataactatctgcgtatctcttattaacctttgttgaagttgatgtcatgttggtttcctagtaatgtcattctggttctgggtctttttctttgttgttttacatgtctgtgttttttttgtcccgttgatgttcctgtttttcccttttggttctcttttttcttaagtatccctgcccctggctctctctcttttttttgtgttcgttctcggctcttgtgtctttctctctgtgtgttttttcagccccctttctctctgcgaggccgttctcaggcttttaaagctagagaaagtcgtgtgttcatgccttcagaaatgaggcatgttttgtgagggatcgtgggcatcgtggttcatgatttgctggttgtgtttcttgttgatccggtcttcaagatgagaagatgataaataatgtacgttgaaaataacagcgatttcacccgaaatgattatttttaatttggaccctaaacttctgccattgaacaattaggttcctgatttcattaattaaacaatttcaaattcaattaagtccccaaacttccaattcttcccatgtttgacataattgatccccaaatttataattcaatccttattagtcccatatcaacccaatctttaattcattcattttttattttttatatattatttttggttattacaatttttattgattattttcaacttaaacatgatatgttatgttttcagaaacacgcattaaactcgctcctgcaaatgaaagagacaagcaacagcttcagtggttttgtgatgcatgcaaaggaataccacaggttcgattcacccttgtatacagttcttcttctgtggtcatagagcaaatgacaacttgtatgctacaagtagatgagacaatttttcagctgaagtatttgaatgtgcatttttcctttcctttcattgaaaccatgataacatttgaggactttttttttataacgctataatttccaatgctatccagattccaacataactaatgttagatgctatagccaaataagaagcctccttgagaataggtttgttttggagtccagtagatccatttagaagaggacatgaaaacctccccttacgaacaattctgagccttctaaaatgggaggaatgcatgctgttgtagggaaatttgttggctttaatcgtttatttttgggttattattggttttcatgtcaccttgggtcaaattgaaaactcatattgaagaacaatacatttgtttcagattgggagaaaccaaagcttaaatgtctttaataaattctaattgttgtctcgttgacatgtatttgtatattatgtactggactacttcccttgaggttcatagtcccccttcaagaatttatgagcccacagtaaactcttttgaaatgaacactagtcattatgctttcctttagacagttatgagtcgcttgctactatgtaagtggtagctcatgctcaaactcccctgaaatgcatactagtcactacgctacggttattgctgaagaaatgcatttatttctgattggtgctactctactttaataggtagaaatactctgatcgcaaatccatacgaactgctgattgaggttcctcttgcacctgattaattgtttcatatttaaaattctagcagttgatttgcttcctttaaaaaaggttttttacttaaatatgcagaaaaggatcctaatgtcacaagtgtatctgtttggttgtgatctgttatagtccaaattctgataagcttttcaagagtgttgcatacctttaaattgaaattcccttgttgtttacatatctatgtatagagtgggaaattttaatgtaaaaacttgcatagtcaataagagctaatatcatttagtcaaagactttgttcattacaagaggacttgtgatacgaatgcctataatgttacaggtaatgcatcctctagaatttctgtccagatctcagaatctggttgttcaagcaactgctccaccatcggatgaagatccagagatagcaatggcagtaaatgcctccattcagtctgccatggcagagcaacctatttttgatacccactctagcactggagcaagttcatcctctagctggaattgccctgttaatgatggcggtcaaggtgctatggatgtgccagcaacacctcctccaaaaacaactagcagcggatgggcaccttatgaaagtgtatctagtggcagctcaactcagcaaaccaagatcttgaatagcagcgttgctgatgtccagacagcaactgatgcccaagattcagtcccatcagctccacccattgtagatgagcttatagaagatggtcccattcactatccatcaattgattccagcccccttgatttttcttccctgccccttgagaatttaccagaaaacacgggtgaaaagaaagaagatggaggttcatcatcatgtgtaatatgtttagatgctccagttgagggagcttgcattccatgtggccacatggttggatgtatgtcttgtttgaaggaaatcaaagccaaggaatggggttgccctgtgtgtcgtgccacaattaaccaggttgtaaggctctatgctgtttgattcttgctgaagaatgaaggtataaatggtaagtattttctgggcaccatagacaattatgttctctttcctatagaacagtatatgaccttaactttcttgtctgaattattaaacaagtataccttagtgtttgtgctttcagtagcatcactgaaatcccctatagtttgaagctaatcatcctccttccgccgatgtagatcttacacttgctacattttgggaattggaattattctctcagtatctaaatcacacaatttaatgctctcaaaactgttaggaagtggttgcattttcattgggaatagcttgattctagttttttgggagtctagtgatttactgtgagactacaagggagtctcttgatctccttataaattttaatctaggatcctgatgagagtaaaatttactagaaacaaaaatgctaaattgctggcaaccgtttctgcagggaagtttggcagcgcaagagagttgacaacttgagttgctgcagttgggcaacttcttggaacattttactcgcttacttgtgattagtaataccatgtatgggttctttaagggtatgccttatgtgagccttttgtgtataatttctttgtatacataagattccaatgtgcttgtatatgaagtcatatatgaactgcgtaaaagggattcaatgcttttgcaattttcaaggtaatcatttatgaactaattctatacac

SEQ ID NO: 4 - protein, 507 aa, Populus trichocarpa
MKFFSIIGNSFGCSASGERLVSAARDGDIQEAKALLEYNPRLARYSTFGVRNSPLHYSAAQGHHEIVSLLLESGVDINLRNYRGQTALMQAGQHGHWEVVLTLMLFKANIHRADYLNGGTALHLAALNGHSRCIRLLLADYIPSIADCWDILSKGSRDNGSISEFDGSALREVINRPADGGITALHMAALNGHVESVLLLLDLGASAYEVTVEDGTTIDLIGAGSTALHYAACGGNPQCCQILIARGASLTAENAKGWTPLMVARSWHRNELEVILTTQQENQSQICPSPYVSIPFMSIVKIARECGWRNNDSLPTCDDTCVVCLETKCTVAAEGCGHEFCTRCALYLCSTICISTVAQGPTGSVACPLCRHGIVSFVKLPGTKPVVKAIARTSLSLSFCTCTGEEPDFPSMTTPLRKPDFQCTKISPPSSFRSLSCQKFPSMNFNASRCMGASDTTPLVPCTIDLNPREREVRCSRSRIRRSTSNPERRKSWLSALNQYVTTGTGC

SEQ ID NO: 5 - protein, 509 aa, Populus trichocarpa
MKFLSIMGNSLGCSASGERLVSAARDGDIQEAKALLEYNPRLARYSTFGVRNSPLHYSAAQGHHEIVSLLLESGIDINLRNYRGQTALMQACQHGHWEVVLTLMLFKANIHRADYLNGGTALHLAALNGHSRCIRLLLADYIPSIADCWEILSKGSKDNESISEFDGSALREVINRPADGGITALHMAALNGHVESVQLLLDLGASVYEVTVEDGTTIDLIGAGSTALHYAACGGNSQCCQILIARGANLTAENANGWTPSMVACSWHRNELEEILSSQPGNRSQICPSSYLSIPFMSIVKIARECGWRNNDSLPTCEDACVVCLERKCTVAAEGCRHEFCTRCALYLCSAICTSTVAQGPTGSVACPLCRHGIVSFVKLPGTKPLVKAIARTSLSLSFCTCSGEEQDFTSMKTLLCKPDFQCTRISPLSSSFRSLSCRKFPSMNFNASRCMGTSDTSPSLVPCTIDRNLRECLVRCSRSRIRQSTSNTERRRSWLSALNQYVTTGTGC

SEQ ID NO: 6 - protein, 454 aa, Populus trichocarpa
MGQGLSCAASQEHAFFTAVQLGEFDTVNAMLERDPSLLHQTTYDRQYPLHIAAANGQIEILTMLLERSVDPDMVNRHKQTPLMLAAMHGKISCLKKLIEAGANILKFDSLNGRTCLHYAAYYGHSDCLQAIISAAQSSPVAVSWGYARFVNIRDGRGATPLHLAARQRRPGCVHILLGNGALVCSSTGGYGSPGSTPLHLAARGGSLDCIRALLAWGADRLQRDASGRIPYVVALKHKNGMCAALLNPSSAEPLVWPSPLKFISELNQEAKALLECALMEANREREKNILMEANREREKNILKGSGHSLPSPSHSADGTDYNISEASDTEVCCICFEQVCTIEVQDCGHQMCAQCTLALCCHNKPNPTTACLNPPVCPFCRSTIVRLVVAKIKNCNDADQDIGEIGSPKLRKSRKSRNFSEGSSSFKGLSATFGKMGGRGSGRIAAENEWMDKP

SEQ ID NO: 7 - protein, 511 aa, Populus trichocarpa
MGNSFGCSASGERLVSAARDGDLVEAKMLLDCNPCLAKYSTFGGLNSPLHFAAAKGHNEIVGLLLENGADVNSRNYCGQTALMQACRYGHWEVVQTLLLFRCNVTRADYLNGRTALHFAAVNGHARCIRLVVADFVPSAPFEAMHARIEGDTGDGSSEKNRFDQSALSRFVNKAADGGITALHMVALNGYFDCVQLLLDIHANVSSETFHYGTTMDLIGAGSTPLHYAACGGNLKCCQILLARGASRTTLNCNGWLPVDVARMWGRHWLEPLLATNSESTIPRFPHSNYLALPLLSVLNIARESGLQCSTRSSDDPDICAVCLERACNVAAEGCRHELCVRCALCLCSTCNISSEMVGPTGSIPCPLCRHGIVSFDRLPGSSAKEMKLPLSLGLCAPCMLHPRDMDGKSLACLPEVRKNRVASVSSDFLCPVTCSPFPSVAIPLCTCNDEPCPSLETQEAESQDESSHRSQTSVEQVKVEGPRLEKTSCSSMFWGRRSCSREHQCTSEINA

SEQ ID NO: 8 - protein, 511 aa, Populus trichocarpa
MGNSFGCSASGERLVSAARDGDLVEAKMLLDCNPCLAKYSTFGGLNSPLHFAAAKGHNEIVGLLLENGADVNSRNYCGQTALMQACRYGHWEVVQTLLLFRCNVIRADYLSGRTALHFAAVNGHVRCIRLVVADFVPSAPFEAMHTQMEGDTRDGSSVKNRFDQSALSRFVNKAADGGITALHIAALNGYFDCVQLLLDIHANVSSVTFHYGTTMDLIGAGSTPLHYAACGGSLKCCQILLARGASRMTLNCNGWLPVDVARMWGRHWLEPLLAPNSDSAIPRFPHSNYLSLPLLSVLNIARESGMHCSVSASDDPDICAVCLERACNVAAEGCGHELCVRCALYLCSTCNIPSEMVGPTGSIPCPLCRHGIVSFVRLPGSSAKEMKLPLSLGLCTPCMLHSHDVDGQSPACLPEVRKNRVVSVSSDFLCPVTCSPFPSVAIPLCTCNDGPCPSFEPQEVESQDESSHRPQTSVEQDKMEGPRLEKTSCSSMFWGRRSCSREHQCNSEINT

SEQ ID NO: 9 - protein, 442 aa, Populus trichocarpa
MGQGLSCAASQDHRFFSAVHFGDLDTVNAMLERDPSLLYQTTYDRQYPLHIAAANGQIEILSMLLERSVDPDMVNRQKQTPLMLAAMHGKISCVKKLVEAGANMLKFDSLNGRTCLHFAAYYGHSDCLQAILSAVQSSPVAVSWGYTRFVNIRDGRGATPLHLAARQRRPECVHILLDNGALVCSSTGGYGSPGTTPLHLAARGGSLDCIRELLAWGADRMQRDASGRIPYVVALKYRNGTCAALLNPSSAEPLVWPSPLKFISELNQEAKALLECALMEANREREKNILKGTGYSLPSPSHSDDGTDDNISEASDTELCCICFEQVCTIEVEDCGHQMCAQCTLALCCHNKPNPTTACLTPPVCPFCRSTIARLVVAKMKDCNDADQDIGDVGSPKLRKSRRSLNFSEGSSSFKGLSATFGKMGGRGSGRIAAENEWVDKP

SEQ ID NO: 10 - protein, 499 aa, Populus trichocarpa
MGQSLDSMTQKQSKDELLYQLAIAGNVDAVKALCSEGAILEWIDRDGKTPLIVACMDSGLYNVAKVLIEMGANVNAYRPGRHAGTPLHHAVKRGLEQTVKLLLSSGANALVRNDDCQTALDVARIKGNINIVRTIESHICYFTGWLREFYGPGFLRAFAPQFLSRKIWAAVIPQGSSNPMTPKKLELVIYPSSQDVQPRTVIALWNAEIEEPNFNRPDPELTIFDQSTKTQYKLASANEGDKQQLHWLYDACSGIPQVMLPPMYGNPPTTVPVVGHHTSAEAVGSAMAIGGGSIQSTTEDNPLHPNTHQSSEVINANGWEDPVRGDSHNRWGVTVASTHSEARSSGWMGEAPKEDHNGCAVPNMGPSGSQDHVQTRYDIPPVSETSGGNTASVPSAPSAPPIPDEELDAGPIHYPSFDFSLLDLSVPAIELGASVTSDVNKGGTSSSCIICWEAPVEGACIPCGHMAGCMTCLSEIKAKKGVCPICRSNINQVTRLYAV

SEQ ID NO: 11 - protein, 468 aa, Populus trichocarpa
MGQQQSKDELLYQQVNYGNIGGIKTLCSQGAGLEWIDKEGKTPLILACRNAGTPLHHAAKRGLENTVKLLLSRGANALVTNDDCQTPLQVARAKGHSIVVRAIESHICLFSGWLREFYGPGFLEVLAPRLVSRDVWVVVLPTGSRNPRRPYKLELAIYSRLQDAQPRTIVALWKANLEEMKFHHSDPSVMIVDNSTTSRGRRRRRARCRSLNVRQTQIKLAPANESDKQQLQWFCDACKGIQQVMHPPAFLHNSQAPAVQATAPPSAEDIEIAMAMNAYIQSAAERPIFDPHSSAGASSSTSWSYPVNTGSQVALETPAAPPPKVTISELALHETGATSSSTQQTKIQNSSIADVQTATDAQDSVPSAPPIVDEIVEDGPIHYPSIDSSPIDTSSLSVENLPENTGEKREDGGSSSCVICLDALVEGACIPCGHMAGCMSCLKEIKAKKWGCPVCRAKIDQVVRLYAV